(19) United States
(12) Patent Application Publication
(10) Pub. No.: US 2022/0031176 A1
(43) Pub. Date: Feb. 3, 2022

(54) IMPROVED CALIBRATION FOR MEASURING THE DIRECT CONTINUOUS BLOOD PRESSURE FROM THE PULSE TRANSIT TIME, PULSE WAVE VELOCITY OR INTENSITY OF THE ELECTROCARDIOGRAM

(71) Applicant: Holger REDTEL, Perleberg (DE)

(72) Inventor: Holger REDTEL, Perleberg (DE)

(21) Appl. No.: 17/299,027

(22) PCT Filed: Dec. 5, 2019
(86) PCT No.: PCT/EP2019/083834

§ 371 Date: Jun. 2, 2021

(30) Foreign Application Priority Data
Dec. 5, 2018 (DE) 10 2018 009 457.0

(51) Int. Cl. A61B 5/021; A61B 5/0235; A61B 5/318; A61B 5/024; A61B 5/08; A61B 5/0205; A61B 5/00; A61B 5/145; B60W 60/00; B60W 50/14; H04Q 9/00

(52) U.S. Cl. CPC A61B 5/02125; B60W 2720/12; A61B 5/318; A61B 5/02416; A61B 5/0816; A61B 5/0205; A61B 5/746; A61B 5/14542; B60W 60/0051; B60W 50/14; H04Q 9/00; A61B 2560/0223; A61B 2562/0247; B60W 2540/221; B60W 2720/10; A61B 5/0235

(57) ABSTRACT

The invention relates to measuring blood pressure and the calibration of said measurement. Here, the intention is to specify solutions which facilitate a more accurate and/or less burdensome measurement. To this end, it is proposed to take pressure measurements in certain respiratory states and/or to undertake the calibration by means of pressure variations without physical activity, i.e., on account of respiration or changes in position, and/or separately for systolic pressure and diastolic pressure.

### PRIOR ART

Continuous blood pressure measurements today are used invasively in clinical practice, in order to monitor patients and document the clinical disease course.

In invasive blood pressure measurement, an artery, preferably the radial artery, is cannulated. The arterial cannula is connected to a pressure transducer via a hose. The hose is filled with an electrolyte solution. The pressure in the artery thus travels via the cannula into the hose and reaches the pressure transducer which converts this pressure signal into an electrical signal for display on the monitor.

In order to prevent clogging of the cannulas and the hoses by blood inflow and clot formation, a pressure bag is attached, by means of which electrolyte solution is flushed through the hose and the cannula into the body, as a rule at 3 m L/hour.

In order to damp the signal as little as possible, there should be no air in the system. Therefore, before the use of the invasive blood pressure measurement, the entire system must be thoroughly flushed with electrolyte solution. Moreover, due to the selection of the hose length, resonances of the system forming the coupling to the cardiovascular system can occur. This is expressed by a superposed wave in the measured values of the blood pressure wave and thus distorts the measurement. To overcome this, another hose length must be selected.

An additional source of error is the elevation of the pressure transducer. Said pressure transducer must be fastened at the elevation of the HIP (hydrostatic indifference point), that is to say at the elevation of the heart. Since in daily clinical routine, patients are asked to sit up and then to lie down again, the position adjustment of the pressure transducer is often forgotten. If the pressure transducer is below the HIP, an excessively high blood pressure is measured and vice versa above the HIP.

An invasive blood pressure measurement, like any invasive procedure, is not risk free. Thus, in 1-4% of the examinations, sepsis and infection occur. Hemorrhages can also occur with a probability of 0.5-2.6%. Hematomas are much more common with 14%. However, most commonly, temporary occlusions of the arteries occur with almost 20%. In rare cases critical circulatory disorders occur (0.09%); however, these complications are particularly fatal, since they can lead to amputation or to a functional impairment of limbs.

Due to these risks and the possible complications, the use of invasive blood pressure measurement day in and day out and millions of times is in fact not justifiable. However, since no adequate substitute is available to date, this method is a necessary evil of contemporary medicine.

Therefore, the invention is intended to disclose an alternative to invasive blood pressure measurement.

Systems and measurement devices for noninvasive continuous blood pressure measurement are offered by only a few manufacturers. These measurement devices are not suitable for daily use by just anyone due to the necessary and elaborate calibration steps.

A method for noninvasive continuous blood pressure measurement used today is the determination of the blood pressure from the pulse transit time. A calibration in the resting state and in the stressed state (during or immediately after sports) of the person to be examined is necessary in order to be able to transfer the value of the pulse transit time to usable pairs of values of systole and diastole. This measurement method is indirect, since the pulse transit time is used for the measurement. However, although the pulse transit time has a correlation with the values for the blood pressure, the exact correlation is only valid for one person and also changes over time. Therefore, the correlation between pulse transit time and blood pressure changes from person to person. In addition, the correlation in case of stress reacts differently. Drugs reinforce these uncertainties. For this reason, currently a calibration, as described above, must necessarily be carried out repeatedly. Currently, such a calibration is necessary relatively frequently due to the inaccuracies and in particular of the pressurized blood pressure measurement according to Riva-Rocci. Therefore, the blood pressure can only be estimated via the pulse transit time or the pulse wave velocity. Wherein the pulse wave velocity can be calculated from the pulse transit time, in that the interval between two locally spaced measurement points for the determination of the pulse transit time is divided by the pulse transit time for the determination of the pulse transit time.

Another currently used method for noninvasive continuous blood pressure measurement is based on the work of Jan Periaz. Here, light is radiated through a finger. The light intensity of the transmitted light is dependent on the blood flow and thus varies with the heartbeat. At the same time, the finger is constricted around the light source and the light receiver, but in such a manner that the blood flow remains constant. Therefore, the strength of the constriction must be continuously adjusted using an electronic control system. The pressure of the constriction from the electronic control system is used as starting signal and converted by a calibration into a blood pressure signal. Therefore, this method too is indirect.

Currently, there is no device available on the market capable of direct noninvasive continuous measurement of the blood pressure.

One method used today for direct but discontinuous measurement of a single value of the blood pressure is based on the so-called Riva-Rocci method. Here the upper arm, or the arm at the wrist is squeezed by an air pressure cuff, and signals of the heart pulse are interpreted. Typically, today, the pressure in the cuff is measured. Said pressure does not exhibit rhythmic variations in the case of low filling with air; in the case of moderate filling with air, a signal having the frequency of the heart pulse can be detected, which disappears again with high filling with air. From the points of the air filling or of the air pressure in the cuff at which the signal appears or disappears, the values for the diastole and the systole can be determined. The mode of operation is represented in FIG. 4.

If a continuous noninvasive measurement of the blood pressure is prescribed today, for example, over 24 hours, then an apparatus working according to the Riva-Rocci method is used, which carries out a measurement at regular intervals, for example, every 15 minutes, and stores its values.

A valuable diurnal profile can be derived from these data only under certain conditions. Due to the continual measurements, the day-night rhythm is disturbed; the patient is reminded of the measurement during each measurement, and the veins and lymph vessels are under an enormous load, and in addition the movement of the patient is restricted by the device.

### SUMMARY

The present disclosure relates to a method for, in particular noninvansive and/or continuous and/or dynamic, blood pressure measurement, consisting of a combination of at least two pressurized blood pressure measurements for different respiratory states and/or elevations of the measuring point with respect to the heart, in particular of a blood pressure course measurement and/or with a blood pressure cuff device, and at least two non-pressurized measurements in particular of a continuous measurement of the pulse transit time, of the pulse wave velocity, of the pulse wave contour, of the electrical activity of the heart and/or of the blood pressure, in particular with a conventional blood pressure cuff device, wherein the pressurized and the non-pressurized measurements are carried out on the same living organism, characterized in that later non-pressurized measurements of the blood pressure, of the pulse transit time, of the pulse wave velocity and/or of the pulse wave contour are carried out, and the measured values of the later non-pressurized measurements are converted by means of the data collected in the pressurized blood pressure measurement into at least one blood pressure value.

The present disclosure further relates to a device for, in particular noninvasive and/or continuous and/or dynamic blood pressure measurement, comprising means for carrying out a non-pressurized continuous measurement of the pulse transit time, of the pulse wave velocity, of the pulse wave contour, of the electrical activity of the heart and/or blood pressure, in particular with a conventional blood pressure cuff device, characterized in that the device is configured to receive measured values of at least two pressurized blood pressure measurements for different respiratory states and/or elevations of the measurement point with respect to the heart, in particular of a blood pressure course measurement, to jointly process the received measured values of the pressurized measurements and the non-pressurized measurements for calibration purposes, and to carry out numerous additional non-pressurized measurements of the blood pressure, of the pulse transit time, of the pulse wave velocity and/or of the pulse wave contour, and to convert the numerous additional non-pressurized measurements of the blood pressure, of the pulse transit time, of the pulse wave velocity and/or of the pulse wave contour in each case by means of the calibration obtained into in each case at least one blood pressure value and to output said blood pressure value.

The present disclosure further relates to a device or system, for, in particular noninvasive and/or continuous and/or dynamic blood pressure measurement of at least two pressurized blood pressure measurements for different respiratory states and/or different elevations of the measurement point of the blood pressure measurement with respect to the heart, in particular of a blood pressure course measurement.

The present disclosure further relates to a method for calibrating results of the measurement of the pulse transit time, the pulse wave velocity, the pulse wave contour and/or of the electrical activity of the heart in a living organism for obtaining continuous values of the blood pressure, characterized in that, for at least one blood pressure value, in particular at least two, in particular at least four different blood pressure values, which were taken for, in particular different respiratory states of the living organism, in particular on the basis of the phase of the respiration, a percentage of inspiration or expiration and/or a temporal position in the respiratory cycle, the pulse transit times, the pulse wave velocities, the pulse wave contours and/or the electrical activity of the heart, which are associated, in particular temporally, with the respective blood pressure values, are used and/or collected.

The present disclosure further relates to use of the influence of the respiration and/or of different elevations of the measurement point of a blood pressure measurement with respect to the heart on the blood pressure for the calibration of a measurement of the pulse transit time, of the pulse wave velocity, of the pulse wave contour and/or the electrical activity of the heart, for the computation of a blood pressure from measured values of the pulse transit time, the pulse wave velocity, the pulse wave contour and/or the electrical activity of the heart.

The present disclosure further relates to a method, for, in particular noninvasive and/or continuous and/or dynamic blood pressure measurement, wherein by means of at least one pressure transducer a pressure variation caused by the blood pressure is continuously acquired, wherein at least two blood pressure values are determined from the acquired pressure variations, characterized in that the influence of the respiration on the determined blood pressure values is reduced, in that, from the continuous blood pressure variations, an influence of the respiration on the variation is determined and/or respiratory states are determined and the blood pressure values are derived from the values acquired by means of the pressure transducer, which were acquired for a predetermined and/or identical respiratory state.

The present disclosure further relates to a device, for, in particular noninvasive and/or continuous and/or dynamic blood pressure measurement, comprising at least one pressure transducer for continuous acquisition of a pressure variation caused by the blood pressure, wherein the device is configured to determine and output at least two blood pressure values from the acquired pressures, characterized in that the device is configured to reduce the influence of the respiration on the determined blood pressure values, in that it determines an influence of the respiration on the variation and/or respiratory states from the acquired continuous pressure variations and derives the blood pressure values from the values acquired by means of the pressure transducer, which were acquired for a predetermined and/or identical respiratory state, and in particular uses the values from the values acquired by means of the pressure transducer as the blood pressure values, which were acquired for a predetermined and/or identical respiratory state, as blood pressure values.

The present disclosure further relates to the method, the device, the use or the system summarized above, wherein the pulse transit time, the pulse wave velocity, the pulse wave contour and/or the electrical activity of the heart for the different parts of the pulse pressure wave such as, for example, the diastole, the systole or the reflection wave, are determined independently of one another, used and/or calibrated to the blood pressure, and/or wherein the pulse transit time, the pulse wave velocity, the pulse wave contour and/or the electrical activity of the heart is/are determined by means of an air pressure cuff, a plethysmography unit, an ECG and/or a pressure sensor.

The present disclosure further relates to the method, the device, the use or the system summarized above, wherein the pressurized calibration measurements and/or blood pressure measurements and non-pressurized calibration measurements, in particular of the pulse transit time, the pulse wave velocity, the pulse wave contour and/or the electrical activity of the heart, occur temporally close together, simultaneously and/or for a similar respiratory state and/or wherein the pressurized calibration measurements and/or blood pressure measurements and non-pressurized calibration measurements, in particular of the pulse transit time, the pulse wave velocity and/or the pulse wave contour occur at different points on the body of the living organism, wherein the points are selected so that a blood vessel extending from or to the heart successively reaches the points.

The present disclosure further relates to the method, the device, the use or the system summarized above, wherein, after a calibration, the pressurization is relaxed and further non-pressurized measurements, in particular of the pulse transit time, the pulse wave velocity, the pulse wave contour and/or the electrical activity of the heart are carried out, for, in particular at least 30 min, for, in particular at least 1 hour, for, in particular at least 6 hours, for, in particular at least 12 hours, for, in particular at least 24 hours, in particular at least every five minutes, in particular at least every two minutes, in particular at least every 60 seconds, in particular at least every 20 seconds.

The present disclosure further relates to the method, the device, the use or the system summarized above, wherein a change in the position of the measurement point with respect to the HIP and/or to the heart is acquired by a position and/or acceleration sensor and used for, in particular the correction of the measurements.

The present disclosure further relates to the method, the device, the use, or the system summarized above, wherein the pulse transit time is determined from the pulse wave contour.

The present disclosure further relates to the method, the device, the use or the system summarized above, wherein, for the pressurized blood pressure measurement, an air pressure cuff is used, which comprises a sensor for the determination of the arm diameter, in particular a bend sensor, a capacitive and/or inductive sensor and/or a sensor which is based on capacitive touch technology, and/or wherein the air pressure cuff is designed so that it is closed stepwise and/or elements are introduced into the air pressure cuff at regular intervals, which can unequivocally be identified by the sensor, and/or in that an air sac of the air pressure cuff is subdivided into multiple chambers, and, in particular, an active surface, in particular an application surface, of the air sac of the air pressure cuff can be adapted to the arm diameter by connecting or disconnecting chambers by means of electrically switchable valves, wherein non-connected chambers are not filled with air during the measurement and/or the air pressure cuff is designed so that an active surface, in particular an application surface, of the air sac of the air pressure cuff can be adjusted by two chambers which are held together in particular by belts, in that a first of the two chambers is used for the blood pressure measurement and a second of the two chambers is used for the deformation of the first chamber, in particular in that said deformation changes the constriction of the first chamber by the belt by means of the pressure change.

The present disclosure further relates to the method, the device, the use or the system summarized above wherein, on the basis of the measurements of cardiac activity, in particular of the blood pressure and/or in particular of the pulse, in particular non-pressured measurements, in particular additional measurements, devices are controlled and/or control instructions and/or handling instructions are output, these devices can be, for example, automated medication systems such as drug pumps, respirators, emergency call systems, transport means, which can transmit an automated emergency call, and/or they can also be autonomously driving transport means, in particular a vehicle, which can autonomously react in particular by detecting critical heart states, in that, in particular, a warning is output to the user and/or, in particular, an emergency call is triggered, in particular the vehicle is driven onto the roadside and/or in particular, a trip to a hospital, in particular to the closest hospital, is initiated, wherein, advantageously, the permitted maximum speed is exceeded, which can be designed to be low-risk in particular by signaling of the vehicle to other traffic participants, in particular by light, sound and/or radio signals.

The present disclosure further relates to the method, the device, the use or the system summarized above, wherein the detection of the respiratory state, of the respiration and/or of the respiration frequency occurs by acquisition of periodic changes of diastole and systole, the interval and/or pattern of the diastole and the systole and/or by acquisition of periodic changes of the pulse pressure and/or by acquisition of periodic changes of the RR interval and/or by acquisition of periodic changes of the oxygen content in the blood, in particular by means of the acquisition of the change of the reflection property of light, for example, by means of a pulse-oximeter or a camera.

The present disclosure further relates to a method for lifesaving and/or protection of traffic participants, wherein, on the basis of measurements of cardiac activity of a passenger of a transport means, in particular an autonomously driven transport means, in particular a vehicle, in particular measurements of the blood pressure and/or in particular of the pulse, in particular non-pressurized measurements, in particular additional measurements, wherein the means used for the measurement communicate, in particular wirelessly, with the transport means, and when critical heart states are detected, an emergency call is transmitted by the transport means and/or a warning is issued to the user and/or an emergency call is triggered and/or the transport means is driven to the shoulder of the traffic lane, and/or a trip to a hospital, in particular to the closest hospital, is initiated and/or carried out, wherein, advantageously, the permitted maximum speed is exceeded, which is designed to be low risk in particular by signaling the vehicle to other traffic participants, in particular by light, sound and/or radio signals.

The present disclosure further relates to a transport means, in particular a vehicle and/or autonomously driven transport means, configured to communicate, in particular wirelessly, with a means for measurements of cardiac activity of a passenger of the transport means, in particular measurements of the blood pressure and/or in particular of the pulse, in particular non-pressurized measurements, in particular additional measurements, wherein the transport means is configured so that, when critical heart states are detected, it transmits an emergency call and/or outputs a warning to the user and/or drives to the shoulder of the traffic lane and/or initiates and/or carries out a trip to a hospital, in particular to the closest hospital, wherein it advantageously exceeds the permitted maximum speed, which is designed to be low-risk, in particular by signaling the transport means to other traffic participants, in particular by light, sound and/or radio signals.

### BRIEF DESCRIPTION OF THE DRAWINGS

FIG. 1 illustrates possible positions and configurations of the invention for the determination of the pulse transit time. Components of the invention are: (A) an air pressure cuff, (B) a computation unit, (C) a plethysmography sensor, (D) an evaluation and representation unit, (E) ECG and (F) a camera.

FIG. 1.a illustrates a configurations where all components are implemented as separated units.

FIG. 1.b illustrates a configurations where the computation unit (B) is integrated in the air pressure cuff (A).

FIG. 1.c illustrates a configurations where the computation unit (B) and the ECG (E) are integrated in the air pressure cuff (A).

FIG. 1.d illustrates a configurations where the computation unit (B) and a second plethysmography sensor (C′) are integrated in the air pressure cuff (A).

FIG. 1.e illustrates a configurations where the computation unit (B), a second plethysmography sensor (C′) and an on device evaluation and representation unit (D′) are integrated in the air pressure cuff (A).

FIG. 1.f illustrates another configurations where the computation unit (B) is integrated in the air pressure cuff (A) and the pulse wave (G) is monitored by a camera (F).

FIG. 1.g illustrates a configurations in which the invention is integrated in a today's intensive care and monitoring station.

FIG. 2 shows examples of data of a measurement which was carried out with a configuration according to FIG. 1.a.

FIG. 3 shows an enlarged section of FIG. 2 which shows that the pulse transit time is dependent on the blood pressure within the heartbeat.

FIG. 4 illustrates the method of Riva-Rocci by showing the course of the air pressure in the cuff during measuring.

FIG. 5 illustrates the source of measurement errors of the Riva-Rocci method due to breathing.

FIG. 6 illustrates the method of Redtel by showing the course of the air pressure in the cuff during measuring.

FIG. 7 shows a comparison of the courses of the blood pressure of a conventional invasive blood pressure measuring system (bottom) and a system based of the Redtel method (top).

FIG. 8 shows is a possible diurnal profile of the systolic value (8.a) and the diastolic value (8.a) of the blood pressure, which can be collected with the improved Redtel method.

FIG. 9 represents different device arrangements and their components for measuring the continuous course of the blood pressure using the improved Redtel method.

FIG. 9.a shows a conventional blood pressure cuff (9.1) enhanced with the new logics of the improved Redtel method (9.2).

FIG. 9.b shows a conventional blood pressure cuff (9.1) enhanced with the new logics of the improved Redtel method (9.2) with the additional usage of force pressure sensors (9.3).

FIG. 9.c shows a conventional blood pressure cuff (9.1) enhanced with the new logics of the improved Redtel method (9.2) with the additional usage of an ECG (9.4).

FIG. 9.d shows a conventional blood pressure cuff (9.1) enhanced with the new logics of the improved Redtel method (9.2) with the additional usage of an ECG (9.4) and a photo-plethysmography unit (9.5).

FIG. 9.e shows a conventional blood pressure cuff (9.1) enhanced with the new logics of the improved Redtel method (9.2) with the additional usage of two different photo-plethysmography units (9.5 and 9.6) at different positions on the body.

FIG. 10 shows an additional representation possibility for a diurnal profile (concerning FIG. 8). This representation possibility is particularly suitable as display in a clock or on the smartphone.

FIG. 11 shows a representation possibility for the blood pressure and its course which was acquired according to the improved Redtel method, and user interaction possibility intended for the (interested) private user.

FIG. 12 shows a typical air pressure course in a continuous blood pressure measurement with a conventional blood pressure cuff enhanced by the logics of the Redtel method but otherwise unmodified.

FIG. 13 shows a typical air pressure course and pressure course in a continuous blood pressure measurement with a conventional blood pressure cuff enhanced by the logics of the improved Redtel method, which also outputs, in addition to the measurement results, data on the time acquisition of the conventional measured systole and diastole.

FIG. 14 shows the enhanced calibration of the Redtel method considering also the influence of breathing, which can for example be determined from a ECG signal.

FIG. 15 shows an additional development stage where the application pressure of the air pressure cuff is to be further reduced.

FIG. 16 shows an additional development stage where the application pressure of the air pressure cuff can be completely removed. This can be achieved with the determination of the pulse transit time which is mapped to a blood pressure value. In this case a plethysmography sensor and an ECG are used.

FIG. 17 shows an additional development stage where the application pressure of the air pressure cuff can be completely removed. This can be achieved with the determination of the pulse transit time which is mapped to a blood pressure value. In this case a two different plethysmography sensors at different body positions are used.

FIG. 18 shows a representation of the spaced measurement by means of a camera. In particular the usage of this method for detection of an occlusive disease in a leg is shown.

FIG. 19 shows a representation of the spaced measurement by means of a camera. In particular the usage of this method for detection of an occlusive disease for example, a stenosis of the carotids, is shown.

FIG. 20 shows a representation of the spaced measurement by means of a camera. In particular the usage of this method for detection of an occlusive diseases in the extremities is shown.

### DESCRIPTION OF THE INVENTION

By means of the invention presented here, these deficiencies should be avoided, and, in particular it should be possible to record a valuable diurnal profile. In addition, a simple and reliable calibration between pressurized blood pressure measurement and non-pressurized measurement, which can be used for a long time, should be made possible.

In addition, the known type of the continuous measurement, based on the Riva-Rocci method, provides insufficient temporal resolution for more in-depth diagnoses. The invention should also disclose how a system for blood pressure measurement that continuously, noninvasively, and directly measures the blood pressure can be constructed. Wherein the temporal resolution enables the determination of a value for the diastole and the systole for each heartbeat.

In the process, the methods presented here not only enable the continuous measurement of the blood pressure but can also be used in order to obtain an improved individual value measurement in comparison to the conventional Riva-Rocci method.

The improvement of the Riva-Rocci method presented here includes in particular both optimizations within the logics and also optimizations of the air pressure cuff.

Current systems for continuous and in particular noninvasive measurement, on the one hand, can be calibrated only with difficulty and therefore cannot be carried out by just anyone, and, on the other hand, the measurement results are based on indirect measurements which can easily be influenced by external factors. Therefore, it should also be disclosed how an easy and automated calibration of the measured values can be implemented.

In an additional configuration, it should also be disclosed how a comfortable, continuous and/or noninvasive long-term measurement, for example, over 24 hours, can be implemented.

An easily operated and pain-free continuous blood pressure measurement device enables new possibilities of application.

Many individuals engaging in leisure sports activities and especially in professional sports today use fitness trackers which, in addition to other non-vital data, can generally only determine the heart pulse data and this often only averaged over a time period. The continuous blood pressure measurement thus enables collecting a much more informative vital value and at the same time it enables collecting the determination of the non-averaged heart pulse for each heartbeat.

By collecting the blood pressure during sports activity or during breaks, the individual engaging in sports can be warned before overexertion, and the risk of an acute cardiovascular disorder (for example, heart attack) during sports can thus be reduced.

Evaluation of the stress state is also a commonly needed application of a measurement system for representing cardiovascular functions. The evaluation here occurs via the measurement and classification of the heart rates and pulse wave variability. In the simplest case, these parameters are obtained from the variation of the heart interval (for example, from the ECG) or from the pulse interval (for example, from the Redtel method (see below) or from a plethysmography) from the current heartbeat to the previous heartbeat. Here, these parameters, as described in numerous other publications, are not identical.

Heart rate variability occurs from beat to beat and is measured directly in the heart, for example, in the form of an ECG. Pulse wave variability is the variability due to a physical situation and is influenced by the changing state of the vessels and of the tissue. Pulse wave variability is determined by the adjustment to the daily life of the tissue and of the vessels and is furthermore determined by the course, the branching, and the state of the arteries. Thus, it differs clearly from the electrical signals measured in the heart, for example, by ECG.

An exact representation of the pressure course in the artery including overnight can moreover be lifesaving for people with risks in the cardiovascular system. Sleep monitoring can be one field of application. The exact recording of the blood pressure curve enables the detection of abnormalities. When such an abnormality is detected, it can be recorded and made available to a physician. Depending on the severity of the abnormality, the patient or a next of kin can be woken up or alerted by a warning signal. Sleep monitoring enables the detection of sleep-related breathing disorders based on the respiration which can be determined based on the respiratory sinus rhythm arrhythmia (RSA) and thus also in the blood pressure course. In addition, measurement of the blood pressure during the night provides a lot of information concerning the risk of developing a cardiovascular disease. Thus, studies show that patients in whom a severe cardiovascular event has occurred present the night before an elevated systolic blood pressure value of on average 7 mm Hg above the values for healthy patients and a value of the diastolic blood pressure that is on average 4 mm Hg lower. This increase in the pulse pressure is also referred to as water-hammer pulse and can be detected using an arrangement according to the invention.

An additional aim of the invention is to improve the blood pressure monitoring in diabetes. In diabetes, high blood pressure can be a symptom, wherein values of the systole above 140 mm Hg are considered to be bad. However, low blood pressures with values of the systole below 105 to 100 mm Hg can also occur. If a high blood pressure, which commonly occurs in type-2 diabetics but which can also occur in type-1 diabetics after several years, is not treated, then it increases during the further course of the disease. High blood pressure values clearly allow the risk of arteriosclerosis, strokes, and heart attacks to increase. Therefore, blood pressure-lowering drugs are administered, which can be associated with side effects. One side effect is that the blood pressure is lowered too much, so that the blood flow through already damaged arteries is no longer ensured.

Continuous blood pressure measurement can reduce the drug intake to a minimum, in that an arrangement according to the invention only recommends a drug intake when the blood pressure increases. During the further course of diabetes disease, the nerves that activate the circulation can be affected. Orthostatic hypotension can occur. This is manifested in that the orthostatic blood pressure dramatically drops and vertigo, light-headedness, blacking out or fainting can occur.

An arrangement according to the invention can detect the precise course of the orthostatic blood pressure and thus quantify the severity of such an ailment.

Current systems for the continuous measurement of the blood pressure are used during surgeries. However, since today these measurements have to be carried out invasively and since this is not risk-free, the measurement is only used in serious surgeries. The blood pressure measurement presented here, due to its noninvasive measurement, can also be used in more minor interventions and thus make them safer.

An additional aim of the invention is to largely replace the invasive measurement as used today in the intensive care station or in the monitoring station.

In an invasive blood pressure measurement, a sensor in the form of a catheter is introduced into an artery of the arm or of the leg. This means that, throughout the measurement, an open wound is present and the patient is confined to the bed. Therefore, a suitable environment, such as, for example, an intensive care station, is necessary for such an examination or monitoring.

In addition, the use of the invasive measurement requires supervision by a physician or by a specially trained care-giving personnel. The invention presented here can also be used by an untrained person at home and causes only minimal pain, wherein at the same time a similar measurement quality is achieved compared to an invasive blood pressure measurement.

FIG. 7 shows a comparison of the measured values of an invasive measurement (7.2) and of the results of the Redtel method (7.1) in a patient, wherein the measurements were carried out simultaneously, wherein the invasive method collected data on the left arm and the Redtel method used the right arm. It can be shown that the pressure amplitude is similar, that the determined RR intervals are similar, and also that abnormalities such as, for example, arrhythmias, can be detected.

In comparison to the invasive method, an additional advantage of the methods presented here, in particular measurement of the blood pressure based on the pulse transit time, is that the selection of the measurement site on the body is not limited and practically any skin surface can be used for the measurement. In the invasive method, large and easily accessible arteries are necessary. Therefore, the radial arteries in the arm are commonly used. If these arteries are no longer accessible, the femoral arteries in the legs or the dorsal foot arteries in the feet can be used. It would be technically possible to use the carotids, but since occlusions can occur due to complications brought about by the invasive measurement, this is not done in practice.

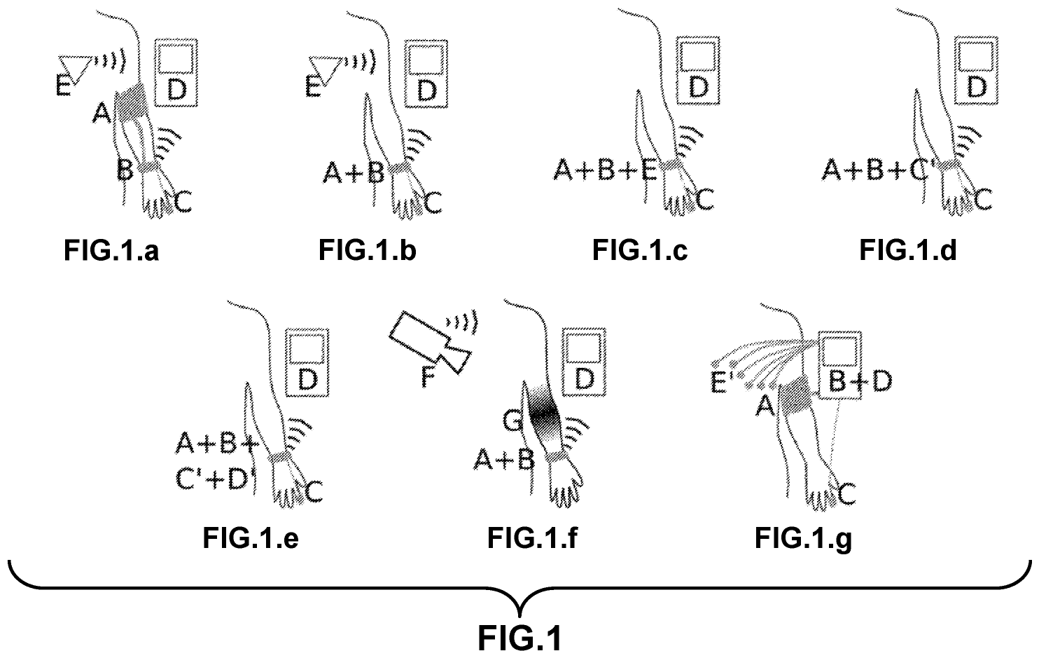

If the invention is integrated into a medical monitoring device, cf. FIG. 1.g and description, then many already currently conventional sensors of such monitoring devices can be used for the invention. This has the advantage, on the one hand, that the existing sensors are already validated systems. On the other hand, a minimal number of sensors is desirable, since the device can be put in operation more rapidly in this manner and the stress for the patient can be reduced.

The determination of the pulse transit time or of the pulse wave velocity can occur, for example, by ECG and plethysmography. Both are conventional sensors in a current medical monitoring device. In addition to plethysmography, a similarly constructed sensor for oximetry measurement may be available; it too enables the accurate representation of the pulse wave.

When plethysmography is used, basically two methods are known for determining the pulse transit time or the pulse wave velocity. The data of a plethysmography show waves whose the troughs and peaks can be associated with functions of the heart. If a second sensor at a distance from the first sensor is used, this can be an additional plethysmography unit, an ECG, a pressure sensor or a system based on the Redtel method; the same functions of the heart can be detected in the signals. The temporal difference between these signals is the pulse transit time which using the spacing between the sensors can be converted into a pulse wave velocity.

The plethysmography sensors, the pressure sensor, and the Redtel method are suitable for the determination of an end time but also for the determination of a starting time. The analysis of the ECG can be used only for the determination of the starting time.

If a sensor is to be used, then the so-called reflection wave can be used. This is a wave which follows the initial wave and which can usually be detected in middle-aged people. The combination of reflection wave and initial wave is referred to as pulse wave contour. The reflection wave can be recorded using the plethysmography unit, a pressure sensor, or the Redtel method. From the time interval between initial wave and reflection wave, the pulse transit time in the aorta can be determined. The pulse wave velocity results from the length of the aortic arc which can be estimated for age and height.

In addition to the use of the pulse transit time or pulse wave velocity, the measured voltage of an electrocardiogram can also be used for determining the blood pressure. Here, the voltages, for example, of the R waves, are analyzed, which correlate with the blood pressure and the respiration. The blood pressure results from the functioning of the heart. Depending on the blood pressure demand, the heart must achieve a stronger or weaker heart pulse. The strength of the heart pulse is given by the voltage signal; therefore, this signal can also be used for the blood pressure measurement and/or blood pressure variation measurement.

In addition to these technical aims, a rethinking in medicine is also sought. Today's medicine is intended to cure diseases. However, a better approach would be to prevent diseases and to increase the quality of life particularly in old age. The methods presented here make it possible to improve two major problems in the field of cardiovascular medicine. On the one hand, critical states can already be detected in the initial stage, whereby a resetting of personal living circumstances can already serve as prevention. On the other hand, in the case of a drug, exactly the correct amount at the correct time can be found.

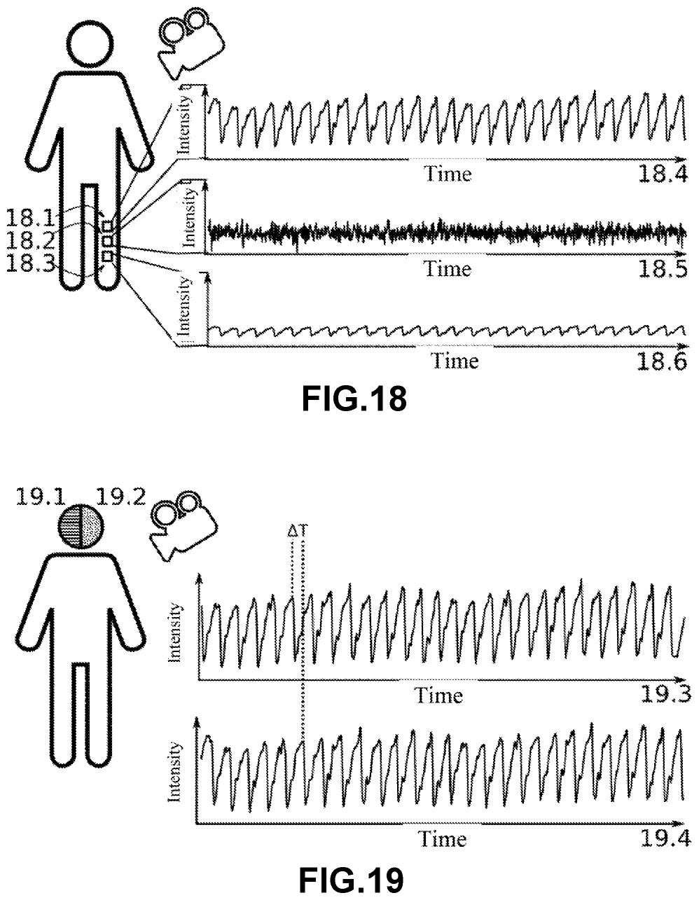

An example of early detection of diseases is occlusive disease (cf. FIG. 18-20). If an occlusive disease occurs in an extremity, the pulse wave velocity changes relative to the healthy extremity. A comparative measurement of the pulse wave velocity in both legs and the observation that these velocities are not the same can thus be an indication of an occlusive disease or preliminary stages thereof. Therefore, a measurement method which is simple to use for measuring the pulse wave velocity is presented.

A medication intake tailored to the situation is necessary for improving the quality of life and can occur better than with the current conventional prescriptions such as, for example, before or after eating. Medication intake cannot be based only on time specifications. It must also include the physical constitution, the age, the height, and the weight. In addition, the measurement of the diurnal profile (cf. FIGS. 8, 10 and 11) enables an additional tailored form for medication intake. If, in the recording of the diurnal profile, an increase of the blood pressure appears and said increase should be treated according to parameters of the treating physician, this can be communicated to the user so that he/she performs a medication. This results in less overdosing of the medication and the intake of the medication occurring at the times when it is also necessary. Other parameters can be, for example, a variation of the pulse transit time or of the pulse pressure.

Today, as a rule, an excessively high dosage of the medication occurs and the blood pressure is set to a fixed value. This setting is in any case not desirable, since it also means that the ability to experience enjoyment is hindered by the possibility of a sudden blood pressure increase; this can result in the occurrence of, for example, depression for example. In addition, an excessively high dosage above the necessary dosage results in side effects, such as, for example, organ damage, beyond the unavoidable side effects. This situation shows that medication intake must be regulated just right, meaning “as much as necessary, as little as possible.” If the blood pressure is acquired continuously, that is to say throughout the entire day, then a slow increase of the blood pressure can be differentiated from an isolated increase. A medication, for example, for preventing high blood pressure can then be initiated when it is necessary and in addition it can also be adapted in terms of its dosage to the pressure difference to be lowered.

For new products in the field of medication, the continuous measurement can be of crucial importance. Thus, new dosage amounts must be developed, which are suitable for bringing about small adjustments of the blood pressure. Since the blood pressure course is known, especially also separately in the diastole and the systole, products can be developed which influence the diastole or the systole independently of one another. Or drugs can be developed which only suppress the dangerous water-hammer pulse and leave the remaining course of the blood pressure unchanged.

An additional problem of current medicine is that all people are treated exactly the same way. Factors such as sex, height, age, and weight are taken into consideration only under certain conditions or not at all. Thus, the limit value for a healthy blood pressure according to WHO 2017 has been lowered. If the systolic value of the blood pressure is higher than 130 mm Hg or the diastolic value is higher 80 mm Hg, this is classified as high blood pressure and thus as pathological. However, these limit values are only appropriate for the average person. An individual having a body height of more than two meters must have a high blood pressure, since otherwise the oxygen supply in the brain is not ensured. Exactly the same applies to older people, in whom the arteries are already calcified. Here too, the high blood pressure is necessary for supplying the brain (and other organs). High blood pressure is not the disease, but rather is the physical reaction to another disease. If the blood pressure is set, undersupply of, for example, the brain occurs, which can result in other diseases such as, for example, dementia.

Exactly the opposite occurs for persons of small stature. A value of 120/80 can already be an indication of a pathological high blood pressure; however, since the limit values do not indicate this, a necessary medication is denied.

These examples show that a rethinking of the blood pressure interpretation is necessary and must be tailored to individuals.

3. The Redtel Method

In the patent specification of DE 10 2018 001 390, PCT/EP2018/056275 and in the DE application 10 2018 007 180.5, in addition to a new measurement device for the pressurized blood pressure measurement, an improvement of the Riva-Rocci method is described, which is carried out on the basis of a physical measurement of a continuous blood pressure measurement, hereafter referred to as “Redtel method.” In the simplest variant, it is disclosed that a continuous measurement is possible, in that new logics for actuating a conventional blood pressure cuff are used.

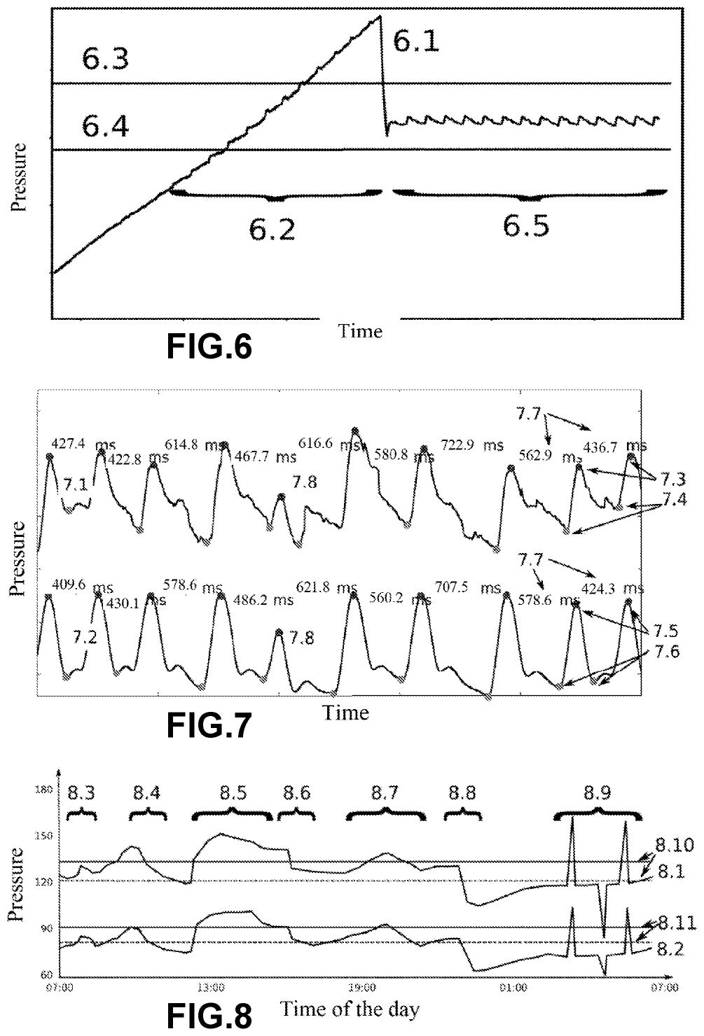

The procedure of a measurement with the “Redtel method” is divided in particular into two phases (cf. FIG. 6). In the first phase, the blood pressure is conventionally measured. The type of the measurement here is not crucial but a measurement according to the Riva-Rocci method is advantageous, since in this way only one device needs to be applied to the body. The values for diastole and systole determined here represent starting values for the logics of the “Redtel method.” In the second phase, the pressure in the blood pressure cuff is adjusted or reduced, and the data of the air pressure in the sleeve are interpreted with the aid of the starting values as continuous blood pressure wave.

This blood pressure wave can be used in order to carry out further analyses. Thus, values for diastole and systole can be determined for each heartbeat based on the starting values, missing heartbeats or too many heartbeats (arrhythmias, cf. FIG. 7) can be detected, or the regularity of the heartbeats can be determined.

This simplest variant is characterized in that the starting values are not used in the measurement procedure according to the Riva-Rocci method.

However, the use of an unchanged Riva-Rocci cuff or in particular an unchanged method has the disadvantage that the calibration is imprecise. The blood pressure curve not only changes with the heartbeat but it is also superposed, for example, by effects of the respiration; this is referred to as respiratory sinus arrhythmia (RSA) (cf. FIG. 5). This explains why the values determined according to the Riva-Rocci method are imprecise. In the Riva-Rocci method, the values for diastole and systole as a rule do not originate from a single heartbeat but can originate from heartbeats that may be offset with respect to one another by up to 60 seconds. In FIG. 5, this situation is shown. The pressure course curve of multiple heartbeats is represented; overall, two breaths are represented. If the measurement is carried out using the Riva-Rocci method (and in particular during the pressure relaxation), the Riva-Rocci method can find different pairs of values (for example, 5.3, 5.4 or 5.5). Here, the values of diastole and systole are temporally far from one another.

Thus, the Riva-Rocci method can yield values which attest that the user is in a healthy state (5.3), a critical state (5.4), or pathological state (5.5) according to the WHO classification, in spite of the fact that the data come from the same pressure course curve.

The blood pressure variation due to the heart pulse is referred to as 1^{st} order blood pressure variation. 2^{nd} order blood pressure variations are triggered by the respiration (and other effects, for example, adjustment of the vessels to outside temperature changes). The 2^{nd} order blood pressure variation influences the blood pressure as a rule for a longer time period than the heartbeat. Studies show that this effect of the respiration accounts for up to 10 mm Hg (Sin P. Y. W., Galletly, D. C., Tzeng, Y. C. Influence of breathing frequency on the pattern of respiratory sinus arrhythmia and blood pressure: old questions revisited, Am J Physiol Heart Circ Physiol 298: H1588-H1599, 2010).

To put this in context, a meta-study (Lewington S., Clarke R., Oizilbash N., Peto R., Collins R. Age-specific relevance of usual blood pressure to vascular mortality: a meta-analysis of individual data for one million adults in 61 prospective studies, Lancet 360: 1903-1913, 2002) shows that an average reduction of the systolic blood pressure by 2 mm Hg already results in a 7% reduction of mortality in coronary heart diseases and in particular a 10% reduction in mortality in stroke cases.

The measurement by conventional blood pressure measurement according to Riva-Rocci has a systematic measurement error of 10%; this means that, in the case of a real systolic pressure of 120 mm Hg, an absolute measurement error of up to 12 mm Hg can occur. An improved calibration according to PCT/EP2018/056275 occurs in particular in that the times when the values for diastole and systole are detected by the Riva-Rocci method are recorded (cf. FIG. 12-17). Since, at the same time, the data for the “Redtel method” are acquired in the background, an accurate calibration can be carried out taking into account the effects of the RSA.

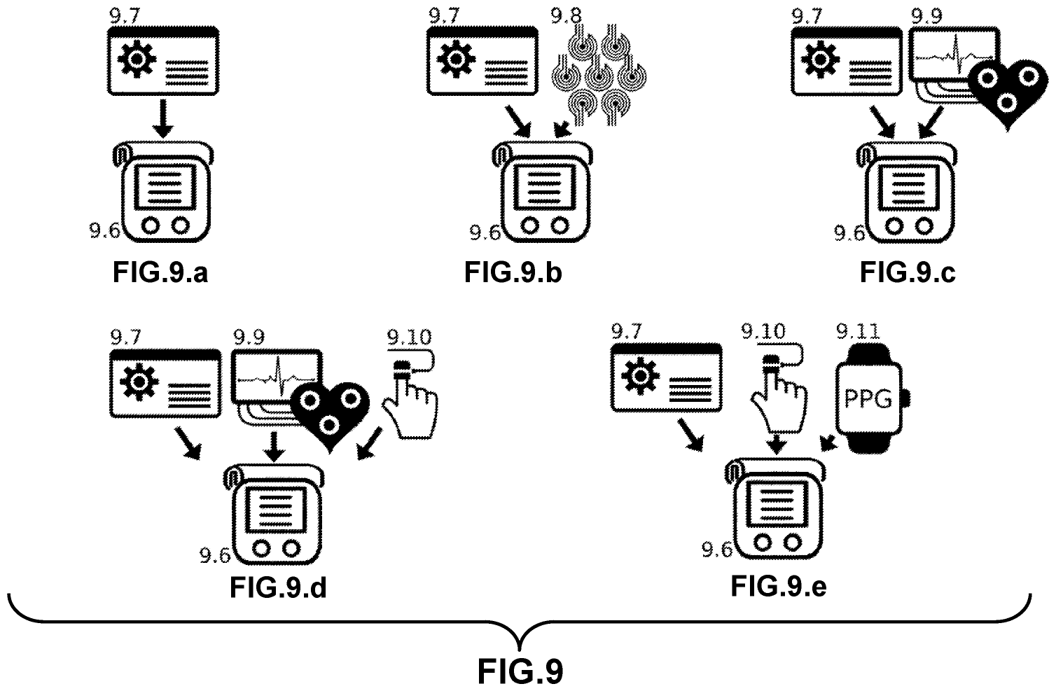

PCT/EP2018/056275 moreover describes how the applied pressure after the blood pressure measurement according to Riva-Rocci is reduced sufficiently so that a blood pressure wave can be determined. Here, the ongoing applied pressure can here be, for example, 100 mm Hg. However, lower pressurizations would be advantageous, since the venous stasis as well as the load on the lymph vessels can only be tolerated for a limited time. For this purpose, as described in PCT/EP2018/056275, new pressure sensors are used, which yield a usable starting signal already at a lower pressurization (cf. FIG. 9.b and 13). The measurement procedure (see also in FIG. 6) with a device according to the “Redtel method” using the integrated variant in a conventional Riva-Rocci cuff is designed in particular as follows: first, a conventional Riva-Rocci measurement is carried out, wherein advantageously the measurement is carried out during the inflation of the cuff. After the cuff has been inflated and the values for systole and diastole are determined, the average pressure in the cuff is reduced, for example, to 100 mm Hg. In the case of such an average pressure, the individual pressure values in the cuff vary with the heartbeat or with the blood pressure variation. With the values for systole and diastole determined from the Riva-Rocci measurement, these variations in the pressure can be associated with variations in the blood pressure, and the representation of the blood pressure curve is possible. By analyzing the individual peaks in this curve, a value for the systole and a value for the diastole can be determined for each heartbeat.

In the following chapter, it is shown, by way of example, how the 2^{nd} order blood pressure variation can be detected, and thus how the systematic measurement error of the conventional Riva-Rocci method can be prevented.

4. Enhancements for the “Redtel Method”

Thus, the following can be stated from the start: The lower the application pressure is, the longer a direct continuous blood pressure measurement can be carried out.

The enhancement of the “Redtel method” consists in that the pressurization can be reduced further, in particular to or under 80 mm Hg or 11 kilopascal, in particular to or under 60 mm Hg or 8 kilopascal, in particular to at least 30 mm Hg or at least 4 kilopascal, so that a long term measurement, for example, over 24 hours, is made possible, so that the conventional long-term measurement based on the conventional Riva-Rocci method can be replaced.

In addition to enabling a continuous measurement, the simple individual measurement via the Riva-Rocci method can also be improved by the logics of the Redtel method.

The improvement of the Redtel method also consists in that the influences of the respiration on the pulse wave and thus on the values of systole and diastole are detected and evaluated and/or the values of diastole and systole at predetermined times within the breath are acquired. The measurement of the blood pressure by the Riva-Rocci method can also be improved, in that the cuff itself is optimized. It is known to the person skilled in the art that, depending on the arm circumference, a suitable cuff should be used in order to achieve an optimal measurement result. The thicker the arm, the wider the cuff should be. Therefore, an improved arrangement of the blood pressure cuff is presented, which makes it possible to use one cuff for all arm sizes, which can be adjusted depending on the arm diameter and thus enables a more accurate measurement.

The aim is achieved, inter alia, by a method for noninvasive continuous blood pressure measurement, consisting of a combination of

at least two pressurized blood pressure measurements fox different respiratory states and/or elevations of the measurement point with respect to the heart, in particular of a blood pressure course measurement and/or using a blood pressure cuff device, and

a non-pressurized continuous measurement of the pulse transit time, of the pulse wave velocity, of the pulse wave contour and/or of the blood pressure, in particular using a conventional blood pressure cuff device,

wherein the pressurized and the non-pressurized measurements are carried out on the same living organism, characterized in that later non-pressurized measurements of the blood pressure, of the pulse transit time, of the pulse wave velocity and/or of the pulse wave contour are carried out and the measured values of the later non-pressurized measurements are converted by means of the data collected in the pressurized blood pressure measurement into at least one blood pressure value.

Pressurized measurement is understood to be in particular a measurement wherein pressure is exerted on the body and/or the blood-conveying vessel, in particular a pressure which is greater than the lymphatic pressure which is typically in the range of 3-5 mm Hg and/or the pressure is greater than 10 mm Hg or 1350 pascal. A non-pressurized or unpressurized measurement is understood to be in particular a measurement wherein no pressure is exerted on the body and/or the blood-conveying vessel, in particular at most a pressure which is lower than the lymphatic pressure which is typically in the range of 3-5 mm Hg and/or the pressure is lower than 10 mm Hg or 1350 pascal, in particular lower than 5 mm Hg or 700 pascal. Here, in particular, the pressure by which the measurement medium or the sensor is pressed onto the skin and/or which is applied to the blood vessel and/or by which the blood vessel is compressed is considered.

According to the invention, for, in particular the calibration, at least two pressurized blood pressure measurements for different respiratory states and/or elevations of the measurement point with respect to the heart are carried out, and at least two non-pressurized measurements, in particular a continuous measurement of the pulse transit time, the pulse wave velocity, the pulse wave contour and/or the blood pressure is carried out. Continuous is understood to mean in particular repetition at the latest every 30 seconds, in particular at the latest every 10 seconds, in particular at least every second, and in particular every half second and/or in particular at least one time, and in particular at least two times per heartbeat. In particular, in a continuous measurement, for each heartbeat, at least two values, for example, the systolic and the diastolic blood pressure, for the blood pressure, the pulse transit time, and/or the pulse wave velocity are determined. Continuous is also understood to mean in particular at least one measurement every X seconds for at least X/10 hours, for, in particular at least X/2 hours. Here X is in particular greater than 0.01 and/or less than 60.

Moreover, according to the invention, subsequently additional non-pressurized measurements of the blood pressure, the blood transit time, the pulse wave velocity and/or the pulse wave contour are carried out. The measured values of the additional non-pressurized measurements are converted by means of the data collected in the pressurized blood pressure measurements and in particular in the non-pressurized measurements carried out for the calibration into at least one blood pressure value, in particular one blood pressure value per additional measurement and/or heartbeat. In particular, since a relationship, in particular a linear relationship, between blood pressure, pulse transit time, pulse wave velocity and/or pulse wave contour is assumed, a conversion can be carried out. Thus, for example, after the calibration by means of at least one non-pressurized optical measurement of the pulse transit time, the pulse wave velocity, and/or the pulse wave contour, at least one blood pressure value can be calculated.

The aim is also achieved, inter alia, by a device for the noninvasive continuous blood pressure measurement, comprising means for carrying out a non-pressurized continuous measurement of pulse transit time, pulse wave velocity and/or pulse wave contour and/or blood pressure, in particular using a conventional blood pressure cuff device, characterized in that the device is configured to receive measured values of at least two pressurized blood pressure measurements for different respiratory states and/or elevations of the measurement point with respect to the heart, in particular of a blood pressure course measurement, and to jointly process the received measured values of the pressurized measurements and the non-pressurized measurements for the purpose for calibration purposes, and

to carry out numerous additional non-pressurized measurements of the blood pressure, the pulse transit time, the pulse wave velocity and/or the pulse wave contour and to convert the numerous additional non-pressurized measurements of the blood pressure, the pulse transit time, the pulse wave velocity and/or the pulse wave contour in each case by means of the calibration obtained into in each case at least one blood pressure value and to output said blood pressure value.

The calibration is repeated in particular at predetermined intervals or intervals determined from measured values, in particular at the earliest after one hour, in particular at the earliest after six hours.

The aim is achieved, inter alia, by a device or system for noninvasive continuous blood pressure measurement consisting of a device according to the invention and means for acquisition and output, in particular to the device according to the invention, of at least two pressurized blood pressure measurements for different respiratory states and/or elevations of the measurement point of the blood pressure measurement with respect to the heart, in particular of a blood pressure course measurement.

The aim is also achieved, inter alia, by a method for the calibration of results of the measurement of the pulse transit time, the pulse wave velocity and/or the pulse wave contour in a living organism, for obtaining continuous values of the blood pressure, characterized in that, for at least two, in particular at least four different blood pressure values, which were taken for, in particular different respiratory states, of the living organism, the pulse transit times, in particular the temporally associated pulse transit times, the pulse wave velocities and/or pulse wave contours are used and/or collected.

The aim is achieved, inter alia, by a use of the influence of the respiration and/or of different elevations of the measurement point of a blood pressure measurement with respect to the heart on the blood pressure for the calibration of a measurement of the pulse transit time, of the pulse wave velocity and/or of the pulse wave contour, for the computation of a blood pressure from measured values of the pulse transit time, the pulse wave velocity and/or the pulse wave contour.

Advantageously, with respect to a method according to the invention, to a device according to the invention, to a use according to the invention or a system according to the invention, the following method and/or following device is/are used for, in particular the pressurized blood pressure measurement, which alone also represent(s) an achievement of the aim.

The aim is achieved, inter alia, by a method, for, in particular noninvasive and/or continuous blood pressure measurement, wherein by means of at least one pressure transducer, a pressure variation caused by the blood pressure is acquired continuously, wherein at least two blood pressure values are determined from the acquired pressure variations, characterized in that the influence of the respiration on the determined blood pressure is reduced, in that, from the continuous blood pressure variations, an influence of the respiration on the variation is determined and/or respiratory states are determined and the blood pressure values are derived from the values acquired by means of the pressure transducer, which were acquired for a predetermined and/or identical respiratory state.

The aim is achieved, inter alia, by a device, for, in particular noninvasive and/or continuous blood pressure measurement, comprising at least one pressure transducer for the continuous acquisition of a pressure variation caused by the blood pressure, wherein the device is configured to determine and output at least two blood pressure values from the acquired blood pressure variations, characterized in that the device is configured to reduce the influence of the respiration on the determined blood pressure values, in that it derives, from the acquired continuous blood pressure variations, it determines an influence of the respiration on the variation and/or respiratory states, and derives the blood pressure values from the values acquired by means of the pressure transducer, which were acquired for a predetermined and/or identical respiratory state, and in particular the values from the values acquired by means of the pressure transducer as the blood pressure values, which were acquired for a predetermined and/or identical respiratory state, as blood pressure values.

Advantageously, with regard to a method according to the invention, to a device according to the invention, to a use according to the invention or to a system according to the invention, the pulse transit time, pulse wave velocity and/or pulse wave contour is/are determined independently of each other for different parts of the pulse pressure wave such as, for example, for the diastole, systole or reflection wave, used and/or calibrated to the blood pressure and/or the pulse transit time, the pulse wave velocity and/or the pulse wave contour is/are determined by means of the air pressure cuff, plethysmography unit and/or the pressure sensor.

Advantageously, with regard to a method according to the invention, to a device according to the invention, to a use according to the invention or to a system according to the invention, the pressurized calibration measurements and/or blood pressure measurements and non-pressurized calibration measurements, in particular measurements of the pulse transit time, pulse wave velocity and/or pulse wave contour, are carried out temporally close together, simultaneously and/or for a similar respiratory state.

Carrying out temporally close together and/or simultaneously relates to the calibration measurements; in particular, it does not cover the additional and/or later measurements which are converted by means of the calibration. However, with regard to all these measurements, it is advantageous to carry out the measurements for a similar respiratory state. Here, different measurement series can and should be carried out for different respiratory states, in particular a separate calibration is carried out for each of these measurement series for the respective respiratory state. As respiratory state, it is possible to use, for example, the indication in percentage of the inhalation amount or time or exhalation amount or time or lung filling. Here, the indication can relate to an absolute maximum or a maximum from a defined measurement time. In the determination of the respiratory state or of a value for a respiratory state, preferably a certain tolerance is applied, because otherwise disproportionately long waiting times can occur until a corresponding correlation of heart function and respiratory cycle occurs (again). This tolerance can be measured using different variables, for example, in percentage of the inhalation and/or aspiration amount, of the duration of a respiratory cycle and/or of the duration of a heartbeat. It is preferable to use a tolerance (absolute span) of at most 1.5 times, in particular at most 1 times the duration of a heartbeat, in particular RR interval and/or 30%, in particular 15% of the inhalation and/or exhalation amount and/or 30%, in particular 15%, of the duration of a respiratory cycle. However, preferably it is attempted here to keep the deviation as small as possible.

Advantageously, with regard to a method according to the invention, a device according to the invention, to a use according to the invention or to a system according to the invention, the pressurized calibration measurements and/or blood pressure measurements and non-pressurized calibration measurements, in particular of the pulse transit time, pulse wave velocity and/or pulse wave contour occur at different points on the body of the living organism, wherein the points are selected in particular so that a blood vessel extending from or to the heart successively reaches the points.

Advantageously, with regard to a method according to the invention, to a device according to the invention, to a use according to the invention or to a system according to the invention, after a calibration, the pressurization is relaxed and additional non-pressurized, in particular continuous, measurements, in particular of the pulse transit time, pulse wave velocity and/or pulse wave contour, are carried out, for, in particular at least 30 min, for, in particular at least 1 hour, for, in particular at least 6 hours, for, in particular at least 12 hours, for, in particular at least 24 hours, in particular at least every five minutes, in particularly at least every two minutes, in particular at least every 60 seconds, in particular at least every 20 seconds, in particular without in the meantime carrying out a pressurization or pressurized measurement.

Advantageously, with regard to a method according to the invention, to a device according to the invention, to a use according to the invention or to a system according to the invention, a change in the position of a measurement point with respect to the HIP and/or with respect to the heart is acquired by a position and/or acceleration sensor and used for, in particular the correction of the measurements.

Advantageously, with regard to a method according to the invention, to a device according to the invention, to a use according to the invention or to a system according to the invention, the pulse transit time is determined from the pulse wave contour.

Advantageously, with regard to a method according to the invention, to a device according to the invention, to a use according to the invention or to a system according to the invention, for the pressurized blood pressure measurement, an air pressure cuff is used, which comprises a sensor for the determination of the arm diameter, in particular a bend sensor, a capacitive and/or inductive sensor and/or a sensor which is based on capacitive touch technology, and/or the air pressure cuff is designed so that it is closed stepwise and/or elements are introduced into the air pressure cuff at regular intervals, which can be unequivocally identified by the sensor, and/or an air sac of the air pressure cuff is subdivided into multiple chambers and, in particular, an active surface, in particular an application surface, of the air sac of the air pressure cuff is adapted to the arm diameter by connecting or disconnecting chambers by means of electrically switchable valves, wherein non-connected chambers are not filled with air during the measurement and/or the air pressure cuff is designed so that an active surface, in particular an application surface, of the air sac of the air pressure cuff can be adjusted by two chambers which are held together in particular by belts, in that a first of the two chambers is used for the blood pressure measurement and a second of the two chambers is used for the deformation of the first chamber, in particular in that said deformation changes the constriction of the first chamber by the belt by means of the pressure change.

Advantageously, with regard to a method according to the invention, to a device according to the invention, to a use according to the invention or to a system according to the invention, on the basis of the measurements of heart actions, in particular of the blood pressure and/or in particular of the pulse, in particular non-pressurized measurements, in particular additional measurements, devices are controlled and/or control instruction and/or handling instructions are output, these devices can be, for example, automated medication systems such as drug pumps, respirators, emergency call systems or also transport means, which can transmit an automated emergency call and/or also autonomously driving transport means, in particular a vehicle, which in particular in the case of critical heart states can react autonomously, in that in particular a warning is output to the user and/or in particular an emergency call is triggered, in particular the vehicle is driven to the roadside and/or a trip to a hospital, in particular to the closest hospital, is initiated, wherein advantageously the permitted maximum speed is exceeded, which can be designed to be low risk in particular by signaling the vehicle to other traffic participants, in particular by light, sound and/or radio signals.

The aim is also achieved by a correspondingly designed and in particular autonomous transport means, in particular a vehicle, as well as by a system consisting of at least one device according to the invention and such a transport means.

A computation unit for the evaluation of the raw measured data can here be arranged, for example, as desired in the transport means and/or in the means for measuring the heart actions. The means for the measurement means can in particular be devices and/or device combinations described in this document.

In addition to the recording of individual values of the blood pressure, a diurnal profile (cf. FIGS. 8, 10 and 11) can be generated. Such a diurnal profile can also be used for the further analysis. Thus, the effects of medication (8.4, 8.6 and 8.8) and whether the medication causes the desired reaction (8.8 in this regard shows an overdosage) are shown. The effects of coffee (8.3), eating (8.5), or sport activities (8.7) can be represented. If abnormalities (for example, 8.9) occur, they can also be represented with good temporal resolution.

The methodology for the generation of the diurnal profile can be used not only for the recording of the state of the cardiovascular system but also for outputting signals for instruction signals to the user (cf. description and FIG. 11). These signals include, for example, the request to take medication (previously agreed on with the physician), to hydrate, to limit sports activity, to regulate the food intake or other user-defined actions. These signals for instruction signals can be output to the user on the basis of the blood pressure situation and they can occur by sound, vibration, or visual representation on a display. Furthermore, information in the form of a (push) message on the smartphone is possible.

Current systems of indirect blood pressure measurement, for example, from Dräger or Somnomedics, use the pulse transit time. These systems must be used with an elaborate calibration in which the person to be examined is first examined at rest and subsequently in the stressed state, that is during or after sports. This already entails a source of error since the resting state and the stress state cannot be achieved with precision or are only vaguely defined. After the calibration, the pulse transit time determined for example by ECG and plethysmography can be associated with a pair of blood pressure values, that is to say, for example, an n-tuple of systolic and diastolic blood pressure.

An improvement of the calibration can also be achieved by using the “Redtel method,” as described in the PCT/EP2018/056275 application.

The goal of the calibration, on the basis of a resting state and a stress state, is to obtain different blood pressures and to examine the effects on the starting values of the plethysmography thereafter. The “Redtel method” also makes it possible to detect minute changes in the blood pressure, which occur naturally, for example, due to RSA (respiration). These minute changes are sufficient for a calibration. Instead of calibration of the blood pressure at rest and under stress, blood pressure values determined with the “Redtel method,” in particular of individual heartbeats during the inhalation and exhalation, can be used. This is possible in particular since multiple pairs of blood pressure values are measured, so that multiple calibrations are also possible. Since the RSA can be detected, an automatic calibration can be carried out, so that no elaborate computation or estimation by the user is necessary. The calibration not only becomes simpler, but it is also more precise and thus generates an improved blood pressure measurement/estimation via the measurement of the pulse transit time. It is also possible to use individual blood pressure values for the calibration and, for example, to calibrate separately for systolic and diastolic blood pressure. Here, different sections of the pulse wave contour can be used and/or different pulse wave contours, pulse transit times, and/or pulse wave velocities can be associated with systolic and diastolic bloods pressure.

5. Further Description of the Invention

The problem of the above-described PCT/EP2018/056275 patent application, referred to here as the “Redtel method,” consists in that a precise continuous blood pressure measurement with the necessary pressurization can be carried out only for a limited time. Venous stasis and much more so the pressure on the lymph vessels prevent a continuous blood pressure measurement over 24 hours. Below, it is shown how the Redtel method can be used in order to measure the blood pressure continuously with precision and over a longer time period with minimal pain for the user.

5.1 Determination of the Blood Pressure from the Pulse Transit Time

The invention described in this patent specification also describes a combination of the methods of the continuous pressurization with a conventional blood pressure cuff, and also by means of a wristband or watch or measurement with the aid of light such as, for example, in a plethysmography unit, or with smart devices such as watches, for example, from Polar, Apple, or also simple smartphone devices, which solve this problem.

The invention consists, for example, of two parts which are either separate or combined in one device.

They are, for example, a means for measuring the blood pressure according to the “Redtel method” and, on the other hand, a means for measurement, in particular unpressurized measurement, of the pulse transit time, pulse wave velocity, and/or pulse wave contour.

The selection of the measuring site for the nonpressurized measurement of the pulse transit time, pulse wave velocity and/or pulse wave contour yields different results which should be interpreted with respect to the position. Starting from the heart, the pulse wave velocity increases with the distance. Thus, in healthy persons the velocity is between 4 and 6 m/s in the aorta and between 10 and 12 m/s in the fingers.

A measurement of the pulse wave velocity is thus always an average of the velocities which exist in the arteries between the measurement points.

For example, the following concrete configurations for the determination of the pulse transit time are possible:

two devices based on the Redtel method, in particular the improved Redtel method

Redtel method, in particular improved Redtel method, and ECG

Redtel method, in particular improved Redtel method, and plethysmography

Redtel method, in particular improved Redtel method, and spaced measurement of the pulse wave

ECG and plethysmography

two plethysmographies.

Some of these possible configurations are represented in FIGS. 1 and 9.

A measurement using light (plethysmography or spaced measurement) requires no pressure application.

The data of the additional measurement for determining the pulse transit time and/or pulse wave velocity and the data of the “Redtel method”, in particular the improved “Redtel method,” are brought together in a computation unit. The data here must be temporally adjustable with respect to one another. Here, the computation unit can also be integrated in a medical monitoring device. The use of other computation units such as, for example, a smartphone is also possible.

This novel continuous blood pressure measurement method according to the invention can be attached on one extremity or on multiple extremities in multiple execution and, for example, can be used for the detection of stenoses or also for the detection of arteriosclerosis. Furthermore, early detection is also possible since the pulse transit time is also changed by vessel stiffness due to plaque deposits which can develop into occlusive diseases.

In a spaced measurement, for example, using a camera, attachments on the body can be dispensed with. In addition, the body can be examined flexibly at different sites; when in shots of sufficiently large areas, a repositioning of the camera can also be dispensed with since any uncovered skin surface accessible for the blood pressure measurement can be measured in its entirety.

5.1.1 Two Devices Based on the Redtel Method, in Particular the Improved Redtel Method

If two devices based on the “Redtel method”, in particular the improved “Redtel method,” are attached on an arm or a leg, then two independent blood pressure waves can be recorded, which, depending on the spacing with respect to one another, exhibit a transit time difference. This transit time difference is the pulse transit time and can be converted using the spacing between the devices into a pulse wave velocity.

The advantage of this combination is that the collected waves correspond to the pressure course in the artery, so that, for each amplitude of the waves, an independent pulse wave velocity, pulse transit time and/or pulse wave contour is/can be determined. If the local minima of the two waves are compared, this results in the pulse transit time, pulse wave velocity and/or pulse wave contour for the diastole; the comparison of the local maxima yields the transit time and/or velocity of the systole. Furthermore, the patterns of the reflection waves can be monitored and thus their velocity can be determined.

The disadvantage of this combination is the short measurement time due to the stress and the limitation of the measurement sites to the limbs.

5.1.2 The Redtel Method, in Particular the Improved Redtel Method, and ECG

Due to the use of ECG, the limitation of the measurement sites can be eliminated. The measurement by ECG yields the pulse transit time from the heart (starting time) to the cuff of the system for the measurement of the blood pressure according to the Redtel method, in particular the improved Redtel method, (end time).

The cuff can be attached as desired to the extremities.

The combination of the Redtel method, in particular the improved Redtel method, with an ECG is described in FIG. 9.c and a typical measurement course is shown in FIG. 14.

In this use, it is advantageous that medically relevant information can be collected in a simple manner.

The pulse transit time can be determined, for example, on the right and left ankles either simultaneously with two cuffs or successively with one cuff.

If the result yields differences in the transit time between two extremities, this is an indication of an occlusive disease or the development thereof, such as, for example, arteriosclerosis, or a stenosis.

The use of an ECG is advantageous since not only can the pulse transit time can be determined but also a better measurement with the Riva-Rocci measurement can be carried out in that the respiration is taken into account.

If the respiration is known, the values of diastole and systole can be associated with the respiratory phase and an improved calibration is possible.

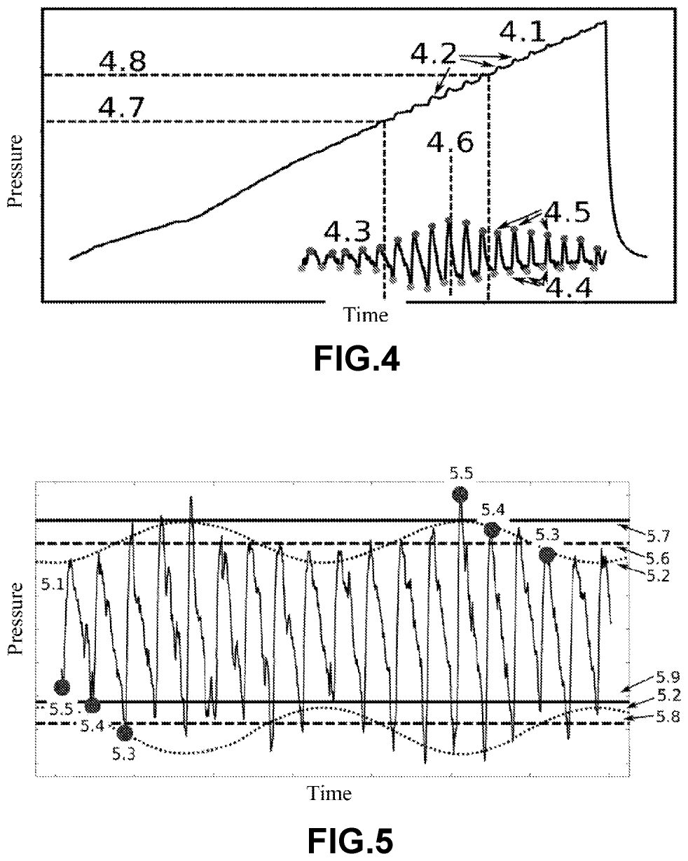

If an arrangement without ECG based only on the Redtel method, in particular the improved Redtel method, is used, then the respiration can also be detected already during a Riva-Rocci measurement. FIG. 4 and FIG. 12 show typical pressure courses in the cuff during a measurement. During the measurement using the Riva-Rocci method, small variations on the rising pressure course curve can be detected. They originate from the cardiac activity and the pulse interval length can here also be determined for each heartbeat. The variation of the pulse interval length originates from the respiration (RSA—respiratory sinus arrhythmia) and in this way the respiration can be detected.

However, the analysis of the ECG with respect to respiration is advantageous, cf. FIG. 14. Here too, the heart interval length from, for example, R wave to R wave for each pulse is determined; the result is the RR intervals. The variation between the individual RR intervals originates from the respiration, so that the respiration can be determined.

However, the measurement using the Riva-Rocci method determines the values for diastole and systole randomly during respiration (cf. FIG. 5). The improvement of the measurement then occurs, for example, in that this situation is recorded in a protocol (cf. FIG. 12-17). Not only the values for diastole and systole but also their measurement times are determined using the Riva-Rocci measurement. Thus, an association of the values of diastole and systole with the respiratory phase is possible. When the Redtel method, in particular the improved Redtel method, is then calibrated with the data of the Riva-Rocci method taking into account the respiration, an improved calibration can be achieved. For this purpose, the values for the diastole/systole in the same respiratory phase as in the Riva-Rocci measurement, with the (starting) measurement values of the Redtel method, are used for the calibration.

Reasoning backwards, this also means that the measurement by the Riva-Rocci method has been improved. An individual value measurement, similar to the current measurement using the Riva-Rocci method, would proceed as follows:

Measurement by means of (current) Riva-Rocci method, followed by calibration of the Redtel method taking into account the respiration and measurement using the Redtel method (including calibration phase) of at least one breath.

Then, for each heartbeat in the measurement phase, a pair of values of diastole and systole can be determined.

Easily represented results would be, for example, the highest systole and the lowest diastole during breathing, the values with the highest pulse pressure or the values of systole and diastole at a fixed point within the phase of the respiratory phase.

5.1.3 Redtel Method, in Particular Improved Redtel Method, and Plethysmography

However, in addition, the plethysmography sensor alone can also be used for the blood pressure measurement. A plethysmography sensor radiates light into the tissue, wherein a portion of the light is scattered and reflected back to the sensor. The intensity of the backscattered light is acquired with a light sensor. Here the light intensity depends on numerous factors, including the blood pressure. If the absolute intensity of the backscattered light is determined, the blood pressure can be determined by means of a calibration. If the relative change of intensity of the backscattered light is determined, a pulse wave contour can be acquired, from which the blood pressure can also be determined by means of a calibration. In such an arrangement, only the device for the “Redtel method” and a plethysmography sensor are therefore necessary (cf. FIG. 1.a and 1.b).

The plethysmography unit can here be used, for example, in two ways. On the one hand, the pulse transit time between the site of the Redtel method, in particular the improved Redtel method, and the site of the plethysmography can be determined, resulting here in the pulse transit time from the system for the measurement of the blood pressure (for example, upper arm) according to the Redtel method, in particular the improved Redtel method, (starting time) to the site of the plethysmography, which is usually on the finger (end time).

On the other hand, the measurement values of the plethysmography can be used in order to reproduce the pulse pressure curve.

5.1.4 Redtel Method, in Particular Improved Redtel Method, and Spaced Measurement of the Pulse Wave

In the spaced measurement, from individual images or image series, for example, “live” videos, on the basis of minute color changes of the skin, invisible to the human eye, a pulse wave is detected. This wave is temporally shifted over the body, depending on the pulse transit time. This allows the analysis with regard to the pulse wave velocity from the pulse transit time and the site on the body.

An exact method of operation of a spaced measurement of the pulse transit time can be gathered from the patent application with reference number DE 10 2018 002 268.5. A possible configuration with spaced measurement is reproduced in FIG. 1.f.

In this method, the pulse transit time can be determined for each point on the body surface and therefore an average over a distance is also not necessary.

The area-comprehensive measurement of the pulse transit time or pulse wave velocity also makes it possible to area-comprehensively determine the blood pressure by a calibration.

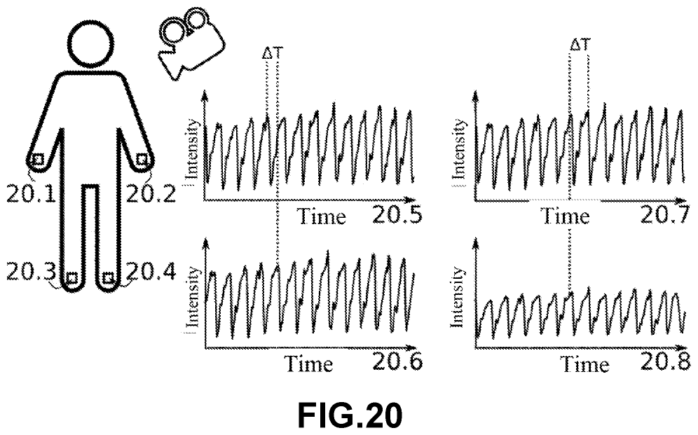

Differences in the blood pressure, in the pulse wave velocity or in the pulse pressure are indications of diseases or development thereof, such as, for example, stenoses (cf. FIG. 19) or arteriosclerosis (FIGS. 18 and 20) whose sites can be located accurately and whose severity can be locally estimated by the pressure difference before and after the occlusive disease (cf. FIG. 18).

An arrangement using a stationary camera is possible; however, it is advantageous to use a smartphone camera. In such an arrangement, the smartphone is the central control unit and is at the same time responsible for the spaced measurement. The data of other measurement devices for the representation of cardiac functions and/or for carrying out a pressurized measurement, such as, for example, of the Redtel method, in particular the improved Redtel method, or of an ECG are transmitted via radio, sound and/or optically and/or by wire connection to the smartphone.

This arrangement makes it possible to simply detect, for example, occlusive diseases. For example, a device for the pressurized blood pressure measurement, in particular measurement according to the Redtel method, in particular the improved Redtel method, is attached on the wrist, for example. This enables the calibration of the measured pulse transit time to the blood pressure. With the smartphone, potential regions are scanned. These regions can be, for example, the legs and feet. Thus, for example, diabetics can check their feet and detect the formation of diabetic foot symptoms early.

Particularly in diabetics, the pulse transit time can be determined for each individual toe. In a healthy person, these transit times are almost identical. In case of an occlusive disease in a toe, this disease can be detected by a different pulse transit time. For the determination of the transit time, the position of the respective image and/or of the measurement point of the pressurized measurement can be acquired, for example, from an image generated by means of the smartphone, by input into the smartphone and/or by a distance measurement, in particular based on radio transmission.

5.1.5 Two or More Measurement Points from a Spaced Measurement

In a spaced measurement, as described in the preceding section, it is possible to measure not only a single point of the skin surface. Instead, each point of the skin acquired in the image, can be measured independently of one another.

For each point, a wave results, which reproduces the pulse course. These waves are shifted with respect to one another due to the pulse transit time. If a point is selected as starting point, then the pulse transit time to any other point can be determined by the shift of the waves with respect to the starting point.

In FIGS. 18-20, different application examples are shown, which have different analysis possibilities. The goal of these analyses is to discover occlusive diseases or detect them even before they manifest themselves.

FIG. 18 shows the detection of a stenosis in the leg. Such an occlusion can be detected in that no pulse can be seen.

But it is useful to detect such a disease not only in the end stage. The development of an occlusive disease starts with the change in vessel stiffness, which occurs, for example, due to plaque deposition. The change in vessel stiffness results in the pulse transit time changing. Therefore, a comparative measurement of the pulse transit time can detect this.

FIG. 19 in this regard shows a measurement of the halves of the face. Due to the supplying carotids, the pulse waves of the halves of the face are temporally shifted with respect to one another. The reason for this is that the two carotids branch off the aorta at different locations. The shift as a rule is 10 ms. if a clearly different value is determined, then this can indicate the development of an occlusive disease.

FIG. 20 shows a similar method for the extremities. If the wave measured in the hand is compared with the wave measured in the foot, then a temporal shift results. In a healthy state of the arteries, this difference should be the same on both sides. If this is not the case, this is again an indication of a developing occlusive disease. This disease can be located in that the waves are analyzed, for example, along the legs (comparable to FIG. 18). The pulse transit time in the leg can be measured proceeding from the thigh, point by point, down to the toes. From the distance from the site of the point on the leg to the site of the first point on the thigh, the pulse wave velocity can be determined. Said pulse wave velocity should increase continuously from the thigh to the toe. If the course on one leg in comparison to the other leg shows one or more discontinuous sites, then these sites are probably the sites associated with a risk of occlusive disease.

5.1.6 ECG and Plethysmography

The measurement of the pulse transit time using ECG and plethysmography is a medically validated method, the results of which can be associated with a blood pressure by calibration. These two measurements have been daily routine in medical practice for decades.

The application here represents an improvement of the calibration (cf. FIG. 9.d and 16).

A current calibration consists in having to set up two different blood pressures in the user, for example, by sports activity. However, based on the teaching of the present application, the variations of the blood pressure due to the respiration (cf. FIG. 5) or due to the movement of the measurement site with respect to the HIP, for example, raising of the hand when the measurement is carried out on the hand, are already sufficient for the calibration.

5.1.7 Two Plethysmographies

In the method presented so far (section 5.1.2), the measurement of the blood pressure was improved in particular by means of the Riva-Rocci method by using an ECG, by means of which the respiration was acquired. However, with the same methodology, this improvement can also be achieved with a plethysmography sensor.

A possible setup is shown in FIG. 9.e, and the mode of operation is described in FIG. 17.

The values of the plethysmography can be examined with respect to the RR interval. As in the ECG, the RR interval varies from beat to beat with the respiration, whereby the frequency of the respiration is determined.

The calibration occurs in particular again with respect to the respiration, as already described in section 5.1.2.

Since the two plethysmography units are attached spaced apart from one another on the body, the two curves are thus shifted with respect to one another due to the pulse transit time.

In addition, the two curves are deformed with respect to one another. This deformation is the result of, besides branching and narrowing of the arteries, the fact that the pulse transit time is a pressure-dependent variable. Thus, the wave component of the systole moves more rapidly than that of the diastole, whereby said wave components separate become increasingly farther apart with distance from the heart.

This also applies to other components of the wave components such as, for example, the reflection wave.

Thus, a pulse transit time for the diastole can be determined independently with respect to a pulse transit time for the systole. Thus, an independent calibration of the blood pressure values, in particular systolic and diastolic blood pressure values, is also possible. Thereby, systolic and diastolic blood pressure can subsequently also be continuously determined independently of one another.

5.1.8 Calibration of a Photo-Plethysmography Unit

The representation of the pulse pressure wave using a plethysmography unit is based on light of a certain wavelength or spectrum being transmitted in tissue. There, the light is partially reflected or absorbed.

Advantageously, a light wavelength is selected, the reflectivity or absorption properties of which in tissue vary as a result of the amount of blood in the arteries. Current systems preferably use the colors infrared, green, red, and blue.

Here, it can be seen that the collected waves at a fixed site on the body are not identical (or respectively linear with respect to one another) and vary with the light wavelength.

Thus, waves recorded with green and blue light have wave crests at sites where a wave recorded with red light has wave troughs.

In addition, it can also be seen that the waves of the Redtel method also differ from the waves of the plethysmography units.

In general, all the waves of the various possibilities for reproducing the heart function differ, at least in detail, and even devices based on the same principle can output different waves.

The use the same light wavelength is appropriate here when two plethysmography units are used, if the units are attached on the body separate from one another. The waves here differ in particular only due to the differences in the blood flow from one measurement site to the next, wherein the detection of these differences is precisely the goal of a measurement.

However, here too it can be advantageous to use two different light wavelengths. If a small size is necessary, for example, a wristband without additional attachments, then the plethysmography units have to be close to one another. The problem then is that, with an identical light wavelength, the signals can be superposed and the two units are then unable to acquire a good signal.

This problem can be counteracted if two different light wavelengths are used; however, then a compensation, a comparison or a calibration between the two different measurements is advantageously necessary.

If a combination of different devices is selected, it should be analyzed first which components of the waves are to be compared with one another in order to determine the pulse transit time.

In principle, there are two procedures for this. One possibility consists in using the two devices on the same site on the body. This results in a pulse transit time of zero. If the two curves are then superposed, the influences to be compared of the heart function on the measurement value waves are also superposed. Then, one looks for points in the cardiac cycle that can be automatically detected. Such points are, for example, local minima and maxima or also inflection points.

The other method is similar but the devices are attached on the sites where they are also located in practice. In addition, the pulse transit time is measured with another already compensated measurement method, wherein the same measurement sites are used. The measurement value waves of the two measurement devices to be compensated are then again superposed and then shifted with respect to one another by the pulse transit time. Again, points that can be found automatically are selected and used for the further computation.

Another possibility of tuning for, in particular plethysmography units, for example, cameras, is the use of color pattern maps. If such a map is filmed or measured by plethysmography, an assignment of a sensitivity of the measurement unit to a light wavelength can occur on the basis of the colors. By means of stored profiles for light wavelengths, a compensation can occur.

If the measurement system is closed, this compensation then has to occur in the development. However, these logics can also be used in open systems. An open system consists, for example, of the combination of a measurement system according to the Redtel method, in particular the improved Redtel method, and a camera of a smartphone. Using the camera of the smartphone, by putting a finger onto the camera and flash, the pulse wave can be determined, in that the flash is at the same time operated in continuous mode, that is to say it is constantly switched on. The light penetrates the finger and, depending on the blood flow, it is absorbed and reflected and then partially reaches the camera. A brightness variation with the pulse wave can be detected. Here, the arrangement of the finger on the smartphone should not be changed.

However, the number of camera and flash modules for smartphones is very large and each module has slightly different properties and sensitivities. Therefore, compensation of the devices by the user is also reasonable.

5.1.9 Calibration of the Pulse Transit Time to the Blood Pressure

For the calibration of the pulse transit time for the determination of the blood pressure with respect from the pulse transit time, the pulse transit time for different known blood pressures is necessary.

Current methods make it possible for the person undergoing the measurement to engage in sports activity. The blood pressure and the pulse transit time before and during or shortly after the sports activity are measured. Here, pairs of values of systolic and diastolic pressures are then compared with the respective pulse transit time.

The methods presented here do not require sports activity since the blood pressure continually changes naturally and this can be detected by the Redtel method, in particular the improved Redtel method.

The blood pressure changes due to a change in the elevation of the measurement site. If a measurement is carried out on the forearm and in standing position, the blood pressure and the pulse transit time can be measured in the lowered arm and in the elevated stretched out arm. Due to the change in the height of the measurement site with respect to the HIP, the blood pressure or the pulse transit time varies. Today, it has been demonstrated empirically that the blood pressure for the diastole changes with a_d=0.5 mm Hg/cm and for the systole with a_s=1 mm Hg/cm.

The change in elevation can be determined based on the arm length which can be derived, for example, from the body height. However, advantageously, the methods for the determination of the correct position given in the next chapter are used.

Inhalation and exhalation change the RR interval (RSA=respiratory sinus arrhythmia) or the pulse rate, the blood pressure as well as the pulse pressure.

The inhalation accelerates the RR interval, raises the systole and at the same time lowers the diastole. As a result, the blood pressure increases.

The opposite occurs during exhalation.

The effects of the RSA on the pressure course curve of the blood pressure are shown in FIG. 5.

Advantageously, one does not consider pairs of values of systolic and diastolic pressures but rather considers individual pressure values from the course of the blood pressure curve.

In particular, the system can be calibrated by the measurement of each individual pulse pressure curve for each heart pulse over a short time span, in particular in the range of the duration of a respiratory cycle and/or 1 to 3 respiratory cycles. This occurs in particular without the human error source.

Current methods for the determination of the pulse transit time consider the shift of two waves with respect to one another, as already described, of the ECG signal and the wave of a plethysmography sensor, for example.

This represents an average. However, the pulse transit time is not constant over the entire pulse cycle but changes with the pressure variation within the pulse. This explains, inter alia, why the pressure curves measured on different arteries differ. The point by point comparison of two waves, for example, on the basis of distinctive points, is sufficient in many cases, but a better calibration can occur if multiple distinctive points are compared.

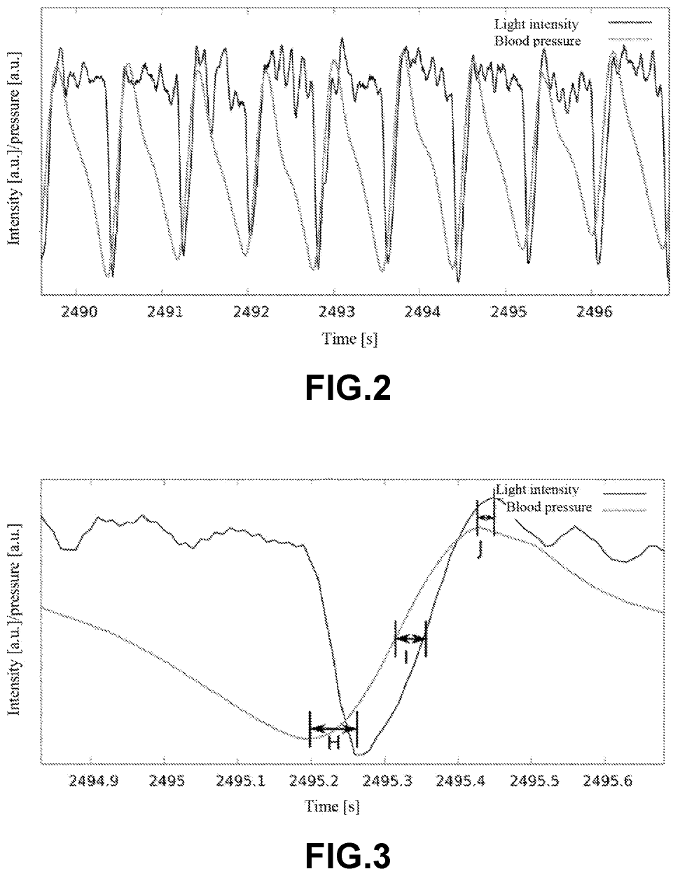

In FIG. 2, by way of example, measurement values of a blood pressure curve which was recorded by means of the improved “Redtel method” and examples of measurement values of a plethysmography sensor are shown. Here, the device which works according to the improved “Redtel method” is attached on the wrist and the plethysmography sensor is attached on the finger of the same hand. In FIG. 3, an enlarged section of the data is shown. It is shown that the pulse transit time decreases with increasing pressure within a heart pulse from diastole (wave trough) to systole (wave crest).

Today, the pulse transit time is a value which is dependent on the measurement site and which ideally is determined for each pulse. Using the “Redtel method,” in particular the improved “Redtel method,” the pulse transit time is then also a continuously acquirable value.

It is medically validated that the pulse wave velocity is linear with respect to the blood pressure values of systole and diastole. However, this relationship is different from person to person and changed by drugs, diseases, fluid and food intake or fluid deficiency and nutritional deficiency and fitness on the day.

Therefore, a calibration is sufficiently accurate only for a limited time period and should be repeated in case of a change.

For a calibration, in particular a linear calibration, at least two different pairs of values of pulse wave velocity and pressure, for example, the values of the systole and diastole of the blood pressure and the pulse wave velocities in the systole and diastole, must be determined. However, to increase the accuracy, it is advantageous to use numerous pairs of values. The pairs of values are in particular linearly approximated and fitted, in particular by regression, in particular linear regression, resulting in the parameters A_s, B_s, A_d, B_d of the linear equations:

S_BD=A_s*PWG+B_s

D_BD=A_d*PWG+B_d,

wherein S_BD is the systolic value, D_BD is the diastolic value and PWG is the pulse wave velocity.

Instead of the pulse wave velocity, the inverse of the pulse transit time can also be used, resulting merely in other parameters (A s, B_s, A_d, B_d) in the linear approximation.

The calibration used today collects a pair of values at rest and a pair of values under stress, wherein the pairs of values in each case consist of three numbers, namely, on the one hand, a number corresponding to the diastolic and systolic pressures and, on the other hand, an (averaged) pulse wave velocity or pulse transit time. Since only two values are therefore present for the calibration, each measurement error has an extreme influence on the quality of the calibration. In addition, no distinction is made between the pulse wave velocity of the diastole and of the systole. The two values, systole, and diastole, today are associated with a single value of the pulse transit time and thus calibrated.

When the pulse wave contours or blood pressure contours are used, a pulse wave velocity and/or pulse transit time can be determined therefrom, in particular using the reflection wave, and then one can proceed as described with regard to the calibration of the pulse wave velocity and/or pulse transit time.

However, the use of the values proposed in this patent specification, which can be present during the RSA and collected for each heart pulse, represent a plurality of pairs of values, whereby the measurement error on a pair of values can have a smaller effect on the result of the calibration. The longer the calibration lasts, the better it becomes; in practice durations in the range of the duration of a respiratory cycle and/or in the range of at least 1 second and/or at most 3 s are usually sufficient.

For the calibration, a value of the pulse transit time or pulse wave velocity for each pulse can be used. However, it is advantageous if the pulse transit time or pulse wave velocity at the time of the systole and at the time of the diastole is used for the calibration and in the subsequent computation of systole and diastole. The pulse transit time is also a variable which continuously changes within the heart pulse, as already described and represented in FIG. 3.

5.1.10 Problem of the Correct Position

In the selection of the compressed air cuff for the Riva-Rocci method, two variants are common today.

Upper arm and wrist blood pressure measurement devices here exhibit a large qualitative difference.

The upper arm can be moved away only to a limited extent with respect to the HIP (hydrostatic indifference point).

The HIP is a point located under the heart. For the estimation of the value, the blood pressure must be determined at this elevation or adjusted to this elevation.

In wrist blood pressure measurement devices, due to movement of the hand with the measurement device, differences of much more than 100 mm Hg above or below the actual blood pressure can result.

This problem in wrist measurements, whether measured physically or with a light, can be compensated for by position and acceleration sensors.

During the calibration, the arm or the hand with the measurement device is moved, for example, from the HIP to the thigh and subsequently raised in a semicircular movement with outstretched arm from the thigh to above the head.

Subsequently there is a return to the HIP. Then the position of the measurement device is calibrated and known. Additional movements can be detected and the value of the blood pressure can be determined knowing the position with respect to the HIP.

A more advantageous determination of the position with respect to the HIP exists if the distance from the HIP to the measurement device is determined and the orientation of the measurement device is known. The orientation results, for example, from the data of an acceleration sensor. If said acceleration sensor is not moved, then it nevertheless indicates an acceleration, namely the acceleration of the earth, and it can thus be detected how the device (at rest) has rotated in space. Since the measurement site on the body is known, for example, on the left wrist with the display on the side of the palm, a limited number of possible positions with respect to the HIP already results from the orientation in space.

Furthermore, the number is limited by the arm length which can be determined via the body height from statistical data.

For a measurement in a conscious user, these data are already sufficient in order to carry out a plausibility verification, in order to give appropriate instructions for the correct use.

However, another aim of this patent specification is to disclose how an arrangement can be used during sleep or in unconscious users.

In order to determine the precise position, an additional device can be used, which is stuck on the chest at a defined site.

This device has one or more radio units and, depending on the design, also one or two ultrasound units; in addition, an acceleration sensor for the determination of the orientation of the additional device is integrated.

The radio unit or respectively the ultrasound unit is used in order to determine the distance from the measurement device. In the case of the use of the radio unit, this can be, for example, a transmitter in the 13.56 MHz ISM band, such as, for example, an RFID unit; for example, the intensity of the radio signal is measured by the measurement unit. Here, the intensity is dependent on the distance between the devices and the distance can be determined.

When ultrasound is used, a prompt is transmitted to the additional device by the measurement device, whereby the additional device outputs a short sound pulse. The measurement device measures the time t between prompt and the arrival of the sound pulse. The resulting distance d obtained with sound speed c is d=c*t.

Two radio units or ultrasound units can be used so that the orientation with respect to the measurement device is measurable.

In addition, the orientation of the additional device must be known; for this purpose, the additional device transmits the data of the acceleration sensor, which at a standstill correspond to the orientation with respect to the surface of the earth.

The distance and the orientation of the measurement device with respect to the additional device can be determined from the distances and the orientation of the additional device with respect to the surface of the earth.

The previous description of the additional device should be used in particular if the user data are incompletely known.

However, if the data of arm length (for example, from a statistic estimation based on the body height), body circumference at different sites, and site of the attachment of the cuff are known and if moreover the type of usage is known (for example, during sleep lying down), a simpler variant can be used.

This variant has only an acceleration sensor which detects the orientation and thus the rotation of the body during sleep.

Via the body circumference, for example, at the shoulder, the elevation of the HIP with respect to the mattress can be determined.

In addition, the measurement device also has an acceleration sensor. The orientation of the measurement device depends on the position of the arm and the position of the arm is limited by the body rotation, so that, from the body rotation, only one possible arm position results, which can give rise to the measured orientation of the measurement device. Via the arm position and the arm length, the elevation above the mattress can also be determined. The difference between the two elevations yields the elevation difference of the measurement device with respect to the HIP.

5.2 Determination of the Pressure Curve from Plethysmography

This calibration presented so far associates a value for the blood pressure with the pulse wave velocity. This calibration is not advantageous for obtaining a representation of the blood pressure wave.

This can be achieved in that the intensity, in particular the absolute intensity, of the light received after transmission of light into the tissue is determined and compared with a blood pressure wave. Since the sites of the light measurement and of the blood pressure measurement as a rule are different, a temporal offset of wave crests and troughs in the pulse pressure waves/curves at the measurement sites is also obtained.

A calibration occurs in that the waves are shifted temporally with respect to one another by the value of the pulse transit time between the measurement points. Depending on the light frequency, the wave can be inverted with respect to the blood pressure wave and display wave troughs in the blood pressure wave instead of at the sites of wave crests.

However, this circumstance is irrelevant for the calibration.

In the context of the possible occurring blood pressures, a linear calibration is possible, so that, by means of the pairs of measurement values, the parameters C and D can be determined in the following equation by linear regression:

P(t-PWL)=C*L(t)+D,

wherein P(t-PWL) is the pressure in the artery at the time when this pressure point passes the site of the cuff and L(t) is the light intensity at the measurement point, P(t-PWL) and L(t) are temporally separated by the value of the pulse transit time (PWL) between the measurement points.

After one calibration or after both calibrations have been carried out, the pressure in the air pressure cuff can be reduced, so that no pressurization is present any longer. In the further course, the blood pressure can be determined via the pulse wave velocity, pulse transit time or via the light intensity by means of the calibration.

If necessary, the calibration can be repeated at any time.

One reason for a repetition can be excessive movement. Another reason can be a change in light intensity beyond the normal variation.

However, a calibration repeated at regular time intervals is advantageous.

5.3 Determination of the Blood Pressure from Electrocardiogram

The absolute value of the voltages of distinctive parts of an ECG is based on numerous factors. Here, the factors can be divided into two groups. On the one hand, the electrodes used, and the hardware used influence the value. In addition, the exact position of the electrodes on the skin and the constitution of the skin can have a great influence on the values of the voltage.

On the other hand, the absolute value of the voltage depends on the current value of other vital parameters. The respiration and the blood pressure should be emphasized.

During a respiratory cycle, the absolute voltage undergoes a periodic variation and increases or decreases. In the case of a poor positioning or poor hardware, the lowering continues to the point that no functions of the heart can be derived from the ECG. This variation with the respiration can be filtered out, in that, for example, only one function of the heart (for example, R wave) is analyzed. Then, in particular during the respiration, one uses only those measurement values which are located approximately (see the aforementioned indications regarding tolerance) or exactly in the same position within the respiration, for example, in the completely inhaled state. As a practicable approximation or alternatively, extreme values selected with respect to a breath can also be used, for example, the maxima of the voltage in a respiratory cycle or the maxima of the voltage in an R wave in a respiratory cycle. This works in particular if the respiratory frequency is small in comparison to the heart rate, which is the case as a rule. Naturally, simultaneously but separately, multiple different functions and positions within the respiration can be analyzed. Then, the variation of the voltage of the selected measurement values in a series, formed by measurement values of a function and with respect to a respiratory state and/or an extreme value in a respiratory cycle in successive respiratory cycles, is dependent only on a possible blood pressure change from one respiratory cycle to the next.

Since with the Redtel method, in particular the improved Redtel method, for each heartbeat, also for each heartbeat in a respiratory cycle, the systolic blood pressure can be determined, the absolute voltage of a cardiac function within a fixed position of the respiratory cycle can be associated with a blood pressure value or calibrated thereto, in particular, first, the blood pressure and simultaneously the ECG are measured. Then, for example, the absolute voltage of the R wave is determined. For example, if said absolute maximum voltage is the highest in a respiratory cycle, then a plurality of such voltages (maximum voltage of the R wave in each respiratory cycle) with the same number of associated systolic blood pressures, in particular linearly, is calibrated, in particular linearly, in particular by regression.

Subsequently, the Redtel method is stopped and the blood pressure can be determined by ECG alone.

5.4 Optimized Arrangement of a Blood Pressure Cuff

For a blood pressure measurement according to the principle of the Riva-Rocci method, an air pressure cuff is placed around a limb. However, the width and length of the cuff should be selected adapted to the arm diameter. The following cuff sizes are currently recommended for the arm diameters

Arm diameter: (width×length)

Less than 24 cm: 10×18 cm

24-32 cm: 12-13×24 cm

33-41 cm: 15×30 cm

More than 41 cm: 18×36 cm

But frequently an incorrect cuff is selected. If too small a cuff is selected, the blood pressure can be overestimated (up to 30 mm Hg are possible) and too high a blood pressure is output.

If the blood pressure cuff is selected to be too large, the blood pressure can be underestimated (errors in the range of 10-30 mm Hg are possible) and too low a blood pressure is output.

An optimization occurs in that this human error in the selection of the cuff is excluded by a technical device. First, by means of an appropriate device, the number of available cuff sizes can be increased and a continuous adjustment can even be achieved.

A current cuff consists of an air sac which is introduced into a fabric so that it can be attached on an arm.

The optimization which represents an independent invention which other described solutions and designs can however also advantageously be combined, is achieved in that a sensor is integrated into the fabric, which can acquire the arm diameter and/or by constructing the air sac out of multiple chambers which can be connected or disconnected from a central sac.

The arm diameter (D) is measured approximately on the basis of the arm circumference (U). The circumference can be converted into a diameter using the circle formula D=U/Pi. Sensors which can determine the arm circumference are, for example, bend sensors which, indirectly via the curvature based on film pressure sensors, change their electrical resistance depending on the bending, wherein the resistance can be associated with a circumference. This sensor can be introduced in any desired manner within the fabric but such that the sensor extends perpendicular to the axis of the limb.

However, due to the mechanical stress of a cuff, it is advantageous to use a sensor or sensor array which does not output an analog value (such as, for example, the change of an electric resistance) but rather outputs discrete values; this is made possible in particular by constructive measures.

The cuff is designed in particular so that only discrete steps of the firmness of the attachment are possible. For example, the cuff can be closed further in steps of one centimeter.

This can occur by means of a perforated strip and a fastener, as in a wristwatch. However, this advantageously occurs, as in current cuffs, in that a portion of the fabric is pulled through a loop and fastened on the cuff by Velcro fastener. Then, in the fabric, at fixed intervals, hard elements or elements that are difficult to deform are introduced, so that the fabric can be pulled through the loop only piece by piece for each element. For each element, a length of the arm circumference is known and it is acquired in particular which elements were pulled through the loop and/or which are not.

In particular in the loop, in the fabric under the loop or in the Velcro fastener, a sensor is introduced. This sensor can be a capacitive and/or inductive sensor or a sensor which uses the “capacitive touch” technology.

In particular, for an inductive sensor, the hard elements are formed from a magnetic material, so that in particular each element has a different and distinguishable magnetization. In a sensor which uses “capacitive touch” technology, metal pieces or fabrics of different size are preferably introduced into the hard elements; in a capacitive sensor, preferably different dielectrics, and/or different numbers of dielectrics and/or dielectrics at different distances from the application surface are arranged in the elements.

If the cuff is closed, a hard element which can be identified by the sensor is located over the sensor, and thus the circumference is determined.

Preferably, the air sac can be/is varied in terms of its active size. For this purpose, the air sac is divided in particular into multiple chambers. These chambers are constructed in particular concentrically around a central chamber. The sizes and number of the chambers can be selected, for example, based on current recommendations. The number of the chambers would then be set at four. The central chamber thus has a size (width×length) of 10×18 cm. This rectangular chamber is surrounded by other chambers which, in the center, have a recess having the size of the small chambers. The outer dimension of the second chamber accordingly is 12-13×24 cm, that of the third chamber is 15×30 cm and that of the fourth chamber is 18×36 cm.

However, it is advantageous to use more than four stages in order to enable a finer adjustment.

The chambers are connected to one another by electrically switchable valves and/or by air lines. In the first case, a valve can connect a chamber to the next smaller chamber, and the central chamber, as in current cuffs, can be connected to the measurement device.

However, the air sac can also be designed so that the active surface can be continuously regulated. Here, the air sac consists in particular of two chambers which are arranged one over the other. The lower chamber is used for the measurement of the blood pressure and the other chamber is used for adjusting the active surface.

The chamber for adjusting the active surface can be filled with air or fluid and is used for the deformation of the lower chamber.

The upper chamber is in particular annular or rectangularly matched to the lower chamber, but with a recess in the center. In addition, the upper chamber is secured, in particular with belts, on the lower chamber. If the upper chamber is inflated, then its change in size is transmitted, in particular by belts, to the lower chamber which is, for example, constricted. The constriction has the effect that the active surface becomes smaller. The degree of constriction can be regulated with the help of the pressure in the upper chamber.

Due to the knowledge of the arm circumference, a solution which does not change the cuff in terms of its function (except for the introduction of the sensor for the measurement of the arm diameter) is possible.

Here, a cuff of average size is used. In the measurement, errors result if the arm diameter does not match the cuff size. However, this type of error is systematic and depends only on the arm diameter. Therefore, it is possible to approximately determine the error and thus to approximately subtract it out of the measurement results.

The measurement error can be determined on the basis of statistically collected lists and determination of the arm circumference or other size features. These lists are prepared for, in particular possible arm diameters or other size features and they contain a corrected result for a possible measurement result.

In a blood pressure measurement, after the closing of the cuff, the arm diameter, or a variable which approximately correlates therewith is determined, and thus, based on an association of arm diameters and suitable cuff size, which is stored in the device, the valves are opened or closed, and/or the measurement values are corrected. This setting of the cuff size is not changed during the blood pressure measurement. A combination of matching and correction is particularly advantageous if the matching occurs/can occur only in steps. The slight deviations from the optimal size can then be corrected in the measured value by computation.

6. Possible Concrete Implementations of the Invention

Possible forms of configuration of the invention are represented purely by way of example and in a non-limiting manner in the purely diagrammatic FIGS. 1 and 9. One configuration in particular always has an air pressure cuff which is operated using the “Redtel method,” in particular the improved “Redtel method.” In addition, an additional unit for the measurement of the pulse transit time or pulse wave velocity is necessary. This can occur by the combination of an ECG with a plethysmography sensor or by means of two plethysmography sensors. However, other methods for the determination of the pulse transit time are also possible, as already described, in particular the air pressure cuff can also be used here, for example, together with an additional device such as ECG or plethysmography sensor.

The following descriptions are intended to disclose possible application fields for the use of the invention as product.

6.1 Fitness Monitoring/Sleep Monitoring

This embodiment is used for the continuous recording of the pulse and irregularities or arrhythmias that occur in the process and for the point by point determination of the blood pressure. Here, the field of use is medical self-monitoring such as, for example, during sports, for example, of performance diagnostics or at night, for example, for monitoring hemodynamic effects of positive pressure ventilation in patients with sleep-related breathing disorder and cardiac insufficiency.

This embodiment of the invention consists of an air pressure cuff for the forearm and a plethysmography sensor which is attached, for example, on the finger. The two devices are connected to a control device on the cuff (cf. FIG. 1.c).

However, it is advantageous to use two plethysmography units, wherein one is located on the finger and one on the wristband and they are connected to the control unit.

Here, the signal of the unit on the wristband is the starting signal and the signal of the plethysmography sensor on the finger is the end signal of a pulse transit time measurement (cf. FIG. 1.d and FIG. 9.e and their mode of operation FIG. 17).

In addition, an acceleration sensor is integrated in the control device. The measurement occurs in that a calibration is carried out using the Riva-Rocci method or the Redtel method, in particular the improved Redtel method. Subsequently, the pressure in the cuff is relaxed and the blood pressure measurement occurs via the light intensity changes of the plethysmography sensor or via the calibrated pulse transit time (cf. FIG. 17). In addition, the plethysmography sensor indicates a wave that reflects the heart pulse, so that the frequency of the pulse or the RR interval and defects, for example, arrhythmias, can be determined.

However, since a movement causes measurement artifacts with respect to the intensity, a continuous blood pressure measurement during the movement can thus usually not be reliably carried out but the frequency of the pulse or the RR interval can be determined.

The integrated acceleration sensor registers movements. When no movement is detected, and the sensor is at the elevation with respect to the heart that was used during the calibration, the blood pressure can be determined. When a position correction is given by an accessory device, the variation of the blood pressure can be compensated based on an elevation with respect to the HIP other than that during the calibration.

The values of the frequency of the pulse or the RR intervals, defects, for example, arrhythmias, and blood pressure are stored and can be read out by means of a radio interface. In addition, a diurnal profile can also be generated (cf. FIGS. 8, 10 and 11).

Warnings can also be output already during the recording of the diurnal profile. During a sports activity, a warning is possible before excessively high blood pressure can occur; for this purpose, the blood pressure measurement can occur in particular during a pause of the sports activity and/or a brief rest of the measurement site, or if the pulse is too low, a motivation can be issued to trigger better performances.

During sleep monitoring, critical situations can be detected, for example, apnea, or heart flutter. It is then possible to react appropriately to these situations, for example, by waking up the person or a next of kin or else by adjusting a respirator or by changing a medication, for example, by means of an infusion pump.

Already today there are systems that combine multiple vital data sensors, for example, a wristwatch manufactured by Omron, “HeartGuide fitness watch.” In this example, the blood pressure measurement according to Riva-Rocci is integrated. By adding the logics of the “Redtel method,” in particular the improved “Redtel method,” to such a watch, said watch can already be able to perform a continuous blood pressure measurement.

According to a communication from Omron, the next version of this watch should also be able to acquire an ECG signal.

By the analysis of the form of the ECG signal, the pulse wave velocity can be determined and thus calibrated to the blood pressure, so that a measurement with low pressurization is possible (cf. the modes of operation FIG. 14 and FIG. 15).

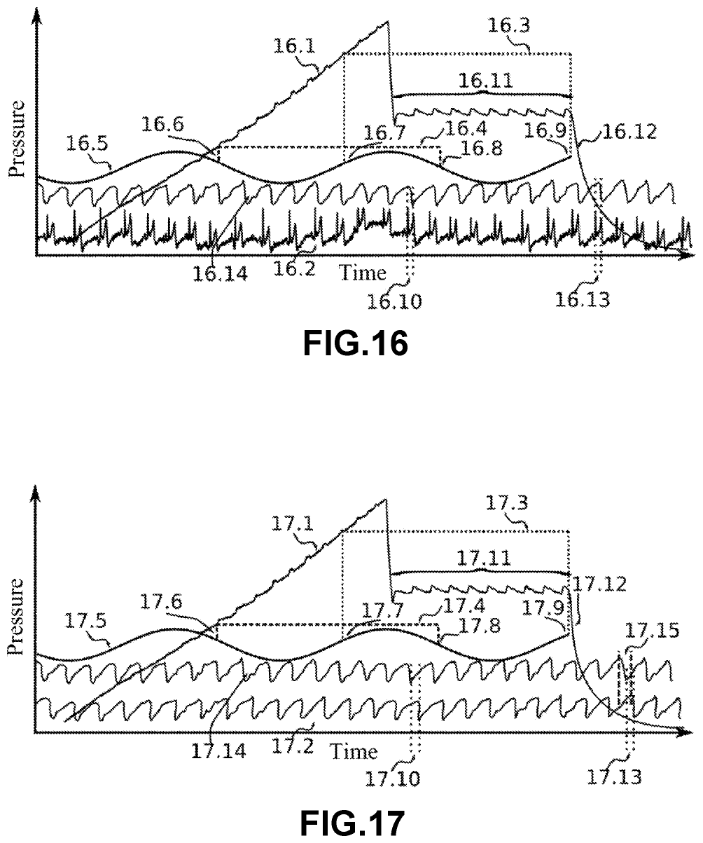

A plethysmography sensor for the determination of the pulse transit time can just as well be integrated in such a watch, as is already the case in numerous other smart watches, resulting in the possibilities for the mode of operation FIG. 16.

The monitoring of the blood pressure and of the heart pulse during the night can be of great therapeutic use.

During the night, apnea can occur. This relates above all to persons suffering from heart disease. By the variations of the blood pressure and of the pulse, based on the RSA, the respiration or its frequency can be detected. In addition, during apnea, phases of elevated and lowered pulse rates also occur.

When apnea occurs, the body frequently cannot enter the rest phase and thus the blood pressure remains at the daytime level and the normal nocturnal lowering of the blood pressure cannot occur.

A respirator can remedy this. This device would assist the respiration if apnea is detected or if apnea is expected based on early symptoms.

For monitoring the blood pressure at night, an intermittent continuous measurement can be carried out.

If an apparatus based on the modes of operation FIGS. 12 to 15 is used, then, during the measurement, the arm is pressurized and a long uninterrupted measurement is not possible without pain. Therefore, an intermittent measurement is reasonable. This intermittent measurement can then consist, for example, in performing a 3-minute measurement every 15 minutes.

If devices based on the calibration of the intensity of plethysmography or ECG are used, then a continuous measurement can be carried out. For a reliable calibration, this continuous measurement is preferably also interrupted here, wherein a new calibration is carried out. This calibration is performed at regular intervals of, for example, 30 minutes, or if excessive movements, measured for example by the acceleration sensor, occur.

If devices which determine the pulse transit time (modes of operation FIG. 16 or 17) are used, a continuous measurement can be achieved.

The calibration of the pulse transit time must also occur at regular intervals but the calibrations can occur at greater time intervals, wherein the required quality of the measurement values determines the calibration intervals. A qualitatively much better measurement than the measurement using the Riva-Rocci method already results with 2-hour calibration intervals. Since the pulse transit time can preferably also be determined during a calibration, a blood pressure can also be determined during the calibration.

6.2 Simplest Short-Term Monitoring/Determination of the Pulse Transit Time

One of the simplest implementations makes it possible to determine the pulse transit time. Here, the embodiment of the invention consists of a Riva-Rocci cuff which also carries out the “Redtel method,” in particular the improved “Redtel method,” and a smartphone which has a camera with a lighting unit.

All current modern smartphones have a camera and a lighting unit (flash).

By touching and covering the camera and in particular the lighting unit with a finger, with the flash switched on as permanent light, light from the flash reaches the camera. Here, the intensity varies with the blood pressure and the pulse. The frequency of the pulse or the RR interval and a pulse wave contour can be determined. The camera, in particular the lighting unit, can also be covered by another body part, resulting in additional measurement possibilities, see below.

The pulse transit time results from the comparison of the blood pressure wave from the cuff and the light intensity wave from the camera of the smartphone. Here the blood pressure wave marks the starting time of a measurement and the light wave marks the end time.

Via the length from the position of the cuff on the body to the position of the camera on the body, the pulse wave velocity can also be indicated.

Light waves in the visible range have different skin penetration depths. Not all light sources thus always radiate the light wavelength necessary for the blood pressure measurement. Smart devices have different light sources. White light in the smartphone suitable for photography contains, for example, light waves from blue 460 nm to red 680 nm.

Smartphone manufacturers such as Samsung use green light, for example in a Gear S3 smart watch.

In order to be able to measure the blood pressure with the smartphone, the light source should be known.

Defined light, for example, 535 nm, makes it possible to calibrate to the minima and maxima of each individual continuous pulse wave.

A possible application is the detection of occlusions of the arteries (stenoses) or of vessel wall calcification (arteriosclerosis). If a large cuff is used, a pressure wave determination can be carried out on the thigh and the measurement of the light source can be carried out on a toe. If this is done for the both legs and different pulse transit times result, this is an indication of an occlusion or vessel wall calcification in a leg.

In principle, if a stenosis exists, the pulse transit time is longer, and in the case of arteriosclerosis, the transit times are shorter in comparison to the healthy state.

6.3 Integration in a Medical Monitoring Device

Current systems for medical circulation monitoring have a plurality of sensors. They include plethysmography sensors or oximetry sensors and sensors for recording the ECG signal standard.

Such systems are offered, for example, by Drager or Siemens.

The blood pressure is determined typically on the basis of one of two methods.

Here the invasive method is in fact very accurate but it requires an intervention into the body and additional external devices in addition to the monitoring device itself, resulting in a limitation of the transportability of the patient, for example, from the accident site to the hospital. Therefore, on medical monitoring devices, there is the possibility of determining the blood pressure via the pulse transit time. This is implemented by ECG and plethysmography. However, to date, suitable calibration can occur only in a few cases, in order to associate an absolute value of the blood pressure with the transit time. As a rule, only variations of the pulse transit time are output as warning.

If then, in addition, a medical monitoring device is equipped with an air pressure cuff and the logics of the “Redtel method,” in particular the improved “Redtel method,” a continuous long-term measurement of the blood pressure can occur.

Here, in particular the pulse transit time is calibrated from the signal of the ECG and of the plethysmography sensor (mode of operation FIG. 16) or from the signals of two plethysmography units (mode of operation FIG. 17). This calibration can occur fully automatically at the touch of a button and requires no human intervention.

The integration also makes it possible to dispense with the invasive measurement since the values of the measurement according to the “Redtel method,” in particular the improved “Redtel method,” are comparable in terms of quality. They exhibit 3-4% deviations with respect to the invasive measurement under optimal conditions and are thus clearly less susceptible to external influences or incorrect operations.

6.4 Remote Diagnosis

The combination of a device for measuring the continuous blood pressure according to the “Redtel method,” in particular the improved “Redtel method,” with a spaced measurement, as can be obtained, for example, in the patent application with reference number DE 10 2018 002 268.5, makes it possible to detect occlusions of arteries.

The spaced measurement can occur in that a transmitted (live) video signal is analyzed. This video signal can be recorded with a stationary camera, but a video recording and analysis with a smartphone are also possible. The results of this measurement are the pulse wave velocity for each skin surface to be detected in the image (cf. FIG. 18).

An occlusion of an artery close to the skin surface has an influence on the pulse wave velocity.

With the aid of a device for the measurement of the continuous blood pressure according to the “Redtel method,” in particular the improved “Redtel method,” which in particular transmits the data, for example, first to a smartphone, which then transmits the data, in particular together with the video signal for analysis, in particular via the Internet, a blood pressure can then be associated with each site on the skin surface on the basis of the pulse wave velocity.

This association makes it possible to estimate the severity of a possible occlusion of arteries by the comparison of the blood pressure before and after the occlusion.

Incipient stenoses and arteriosclerosis can also be detected on the basis of vessel stiffness or due to plaque deposition, which lead to differences in the pulse transit time. For this purpose, comparative analyses are carried out in different body positions and/or halves.

If the right half of the face is compared to the left half of the face, then a difference in the pulse transit time between left side and right side can be detected (cf. FIG. 19). This is due to the length of the aortic arc from which the two carotids branch off at different sites. If the transit time then differs considerably from a normal state (10 ms from the left side to the right side), then this is an indication of stenosis or its development in the carotids.

A similar procedure can be carried out for the detection of arteriosclerosis in the extremities. The pulse wave velocities between the left and right extremity can be analyzed or the pulse wave shifts between the arms and the legs on both sides can be compared (cf. FIG. 20). If a difference between the right side and left side arises, then this is an indication of an occlusive disease or its development. Early recognition is possible since the pulse transit time is already changed due to vessel stiffness, for example, by plaque deposition.

6.5 Additional Fields of Application

Since the configurations of the invention are easy to operate without pain, new possibilities of application arise based on daily use.

Thus the daily routine of a user can be guided.

Most simply, it is a warning to the user or a caregiver in case of cardiovascular problems. If a problem is detected, the user can be alerted to it and then, based on previous diagnosis or instruction of a physician, take drugs or limit one's current activities (for example, sports) and/or change one's current behavior. This is made possible in particular if a diurnal profile (see FIGS. 8, 10 and 11) is recorded.

The continuous monitoring of the blood pressure can allow the patient to take drugs in a manner adapted to the situation. Here, the need and dosage can be determined on the basis of the instantaneous recording and/or of the recorded profile, for example, diurnal profile.

The monitoring of the daily routine makes it possible to better detect the stress and emotional state. Rapidly increasing high blood pressure with low variability and shallow breathing is a sign of stress. In sports activities, an elevated blood pressure is desirable, but if the blood pressure or the pulse frequency rises above a healthy level, this can be detected and prevented in time by a warning.

An additional warning possibility exists for incorrect diet. If a user wishes to lose weight, for example, this is often a process which requires increased will power. The problems are that at first the appetite remains strong and when first progress is made the tissue slowly atrophies, which is usually considered unattractive. To prevent these problems from leading to discontinuation of the endeavor, quantified information on the eating behavior can be helpful. It is helpful to make a game of the endeavor, so that the achievement of levels (for example, soup week completed) and records (for example, 10 consecutive days under 2000 kcal) can have a motivating effect. The invention here makes it possible to document the eating behavior. When food is eaten, the blood pressure increases. This increase depends on the food and the quantity. The more food is eaten, the more the blood pressure increases. The invention would output a warning when an excessively high blood pressure rise occurs while eating, so that the eating can be adjusted in time so as not to jeopardize the endeavor to lose weight.

An additional warning possibility of warning in case of incorrect diet is the detection of fluid deficiency. Especially older people lose the sensation of thirst and therefore they hydrate less. The blood pressure is an indicator of the fluid balance. In case of prolonged fluid deficiency, the blood pressure increases. Therefore, a long-term measurement can be used in order to be able to evaluate quantitatively the compliance with drinking schedules and the quality of care.

In addition to the measurement variable blood pressure, the heart pulse is also an important information value. Abnormal variations of the pulse and of the blood pressure indicate cardiovascular problems. In people in risk groups, a lasting continuous measurement could therefore be used in order to possibly be able to transmit an automatic emergency call.

The same monitoring can also be used in individuals in position where safety is relevant, for example, in passenger transport, or in the operation of machinery.

For example, locomotive conductors could be measured. If a cardiological incident then occurs, for example, a heart attack, the train can be stopped without accident. At the same time, an automatic emergency call for the locomotive conductor can be transmitted and the control center can be informed, so that it can institute further measures such as rerouting of other trains.

In large transport ships, only a minimal crew is used today, which is moreover distributed over the ship as it does its work, so that personal contact occurs only rarely during the day. If a cardiac cardiological incident occurs, this can remain unnoticed by the rest of the crew for many hours. If the crew members can be measured, these incidents can be communicated to the rest of the crew.

Current developments show that in the future of passenger transport may take place with partially or autonomously driving vehicles. In such a vehicle, the detection of cardiac problems can be used so that the vehicle drives to the roadside in time to prevent an accident while simultaneously an automatic emergency call is transmitted. In fully autonomous vehicles, the drive to a nearby hospital can also be initiated. By the communication of the autonomous vehicle with other autonomous vehicles on the road, the vehicle can, like an emergency vehicle, also normally be allowed to drive faster than normal since it has appropriately informed the other vehicles. As an example, an autonomous trip from Berlin to Munich is mentioned. At the beginning of the trip, the destination is entered and the vehicle drives without further user interaction to the destination. If a cardiological incident occurs or the driver dies, the vehicle with the deceased or unconscious driver drives for hours to the destination. Having arrived at the destination, the vehicle autonomously parks in a parking spot, where the driver remains unnoticed for several days. Measuring the driver can therefore be lifesaving since a trip to the hospital in time can be organized, or if this is no longer possible at least a dignified handling of the deceased driver is possible.

### DESCRIPTION OF THE FIGURES

FIG. 1

The figure shows examples of possible positions and configurations of the invention. In a simple configuration (1.a), all the subcomponents are implemented as separate units. The air pressure cuff (A), which operates by means of the computation unit in accordance with the “Redtel method,” in particular the improved “Redtel method,” is connected via a hose to the computation unit (B). In addition, a plethysmography sensor (C) is connected by cable to the computation unit. By radio transmission or wire, the computation unit sends the data of the cuff and of the plethysmography unit to an evaluation and representation unit (D). The evaluation and representation unit can be, for example, a smart device or a medical monitoring device, for example, a Drager monitor. For the determination of the pulse transit time, a separate ECG (E) is used, which sends its data to the same evaluation and representation unit (D).

In an additional configuration (1.b), a forearm cuff device (A+B) can also be used, in which a computation unit is already integrated.

The integration of an ECG into the forearm cuff device (A+B+E) is also possible in an additional configuration (1.c). However, the recording of an ECG signal can only occur if the Cabrera circle is closed, in that a finger of the other hand is put on an outer electrode of the design.

Therefore, in an additional configuration (1.d), it is appropriate to determine the starting time for the determination of the pulse transit time not by means of an ECG signal but via an additional plethysmography sensor which is also integrated in the forearm cuff device (A+B+C′).

The evaluation and representation unit does not necessarily have to be an external unit; thus, in an additional configuration (1.e), the evaluation and representation unit can be integrated in the forearm cuff device (A+B+C′+D′). Here it is possible, although not absolutely necessary, to transmit the data also to an external evaluation and representation unit (D).

In addition to the conventional measurement of the pulse transit time or pulse wave velocity, modern methods can also be used, such as, for example, the analysis of moving images. In an additional configuration (1.f), a camera (F) is directed onto the patient. By a suitable analysis, the pulse wave (G) and its movement can be made visible on the video image. The pulse transit time and the pulse wave velocity can be determined. Thus, a plethysmography sensor and a measurement arrangement for recording the ECG can be dispensed with.

In today's intensive care and monitoring stations, a medical monitoring device which processes data of a wide variety of sensors and displays measured values is used. The invention presented here in an additional arrangement (1.g) can be integrated in such a monitoring device (B+D). The integration is thus advantageous since sensors that are already necessary, such as, for example, a medical ECG (E′), a plethysmography sensor (C) or a cuff (A), can be present.

FIG. 2

The figure shows examples of data of a measurement which was carried out with a device which is provided with the logics of the improved “Redtel method” (blood pressure course), and unprocessed data of a plethysmography sensor (course of the light intensity). The data were acquired with a configuration as represented in FIG. 1.a. wherein the air pressure cuff was attached on the forearm and the plethysmography unit was placed on the finger of the hand.

It can be seen that the curve of the light intensity runs temporally after the curve of the blood pressure, which can be seen from the temporal positions of the minima.

FIG. 3

The representation shows an enlarged section of FIG. 2. It is shown that the pulse transit time is not constant over the pulse. The resulting intervals are as follows: H: 80 ms (wave trough to wave trough or diastole); I: 40 ms (inflection point to inflection point) and J: 20 ms (wave crest to wave crest or systole).

FIG. 4

The pressure course as a function of time (4.1) is shown, which is present in the case of a conventional measurement according to Riva-Rocci in the cuff. Here, the measurement variant with increasing pressure is shown. The pressure course shows small variations (for example, mark 4.2) which are attributed to the heart pulse. If the general pressure increase is subtracted from the pressure curve, the result is the pressure variation which is attributed to the heart pulse alone (4.3). In this curve, local minima (for example, mark 4.4) and local maxima (for example, mark 4.5) are determined. The interval in amplitude between a local minimum and a subsequent local maximum is determined, and the largest interval is found (4.6). Starting from the largest interval, in the direction of temporally earlier intervals, one looks for an interval which corresponds to an empirical percentage of the largest interval. The point in time where this interval is found is the point in time when the diastolic blood pressure (4.7) existed in the cuff. A similar approach is used for the systolic blood pressure (4.8), in that at temporally later intervals, one looks for an interval which has a (different) empirical percentage with respect to the largest interval.

FIG. 5

Representation of the influences of respiration on the measurement results of the Riva-Rocci method. Shown is a temporal course of the arterial blood pressure over two full breaths (5.1) recorded using the improved Redtel method. The curve comprises a pressure variation for each heart pulse, which varies between the diastolic blood pressure value and the systolic blood pressure for this heart pulse. Here, the amplitude of the pressure variations varies with the respiration (shown diagrammatically using lines 5.2).

If a measurement using the Riva-Rocci method is now carried out, values for the diastolic value and the systolic value are determined, which correspond to the local minima and the local maxima. Here, the values for the diastole and the systole, due the measurement method, are temporally separated by several pulse beats.

Examples of possible pairs of values are the marks 5.3, 5.4 and 5.5.

Currently, according to the WHO, the following limit values for the blood pressure apply (indicated by lines):

systole: more than 130 mm Hg: hypertension stage 1 (5.6), more than 140 mm Hg hypertension stage 2 (5.7) diastole more than 80 mm Hg: hypertension stage 1 (5.8), more than 90 mm Hg hypertension stage 2 (5.9).

Accordingly, the measurement point 5.3 would be evaluated as healthy, the measurement point 5.4 as hypertension stage 1, and the measurement point 5.5 as hypertension stage 2.

This example shows that the measurement results of the Riva-Rocci method, in terms of their quality, can in fact not be used for making a precise diagnosis, since different results are determined with the same pressure course.

FIG. 6

Representation of the pressure course in the cuff when the Redtel method (6.1) is used. In the Redtel method, first a measurement (6.2) comparable to the Riva-Rocci method is carried out. However, for an accurate calibration of the improved Redtel method, the measurement times of the determined systole (6.3) and diastole (6.4) are recorded. Subsequently, the pressure in the cuff is reduced, for example, to 100 mm Hg; other values are also possible.

With the values for systole, diastole, and their measurement times, the further course of the pressure in the cuff (6.5) can be calibrated and displayed as result wave and output for further analysis.

FIG. 7

Comparison of pressure curves which were determined using the improved Redtel method (7.1, on the right arm) and using the invasive method (7.2, on the left arm, Drager system). From these curves, the local minima (for example, improved Redtel method 7.4, invasive method 7.6) and the local maxima (for example, improved Redtel method 7.3, invasive method 7.5) were determined. Using these values, it is possible to determine, in addition to the blood pressure, also the RR interval (for example, 7.7) for each heartbeat.

In addition to the vital data, abnormalities such as, for example, an arrhythmia (7.8) can also be detected and represented.

The comparison shows that the improved Redtel method can collect data which are comparable in terms of quality and accuracy to the invasive method, which is the current gold standard of blood pressure measurement.

FIG. 8

Shown is a possible diurnal profile of the systolic value (8.1) and the diastolic value (8.2) of the blood pressure, which can be collected with the improved Redtel method.

In addition, the limit values of the WHO are included in the drawing (see also FIG. 5, systole 8.10, diastole 8.11).

The course shows typical changes of the blood pressure due to everyday situations and due to medication.

8.3: Effect of drinking coffee.

8.4: Intake of blood pressure-lowering drugs

8.5: Lunch

8.6: An additional intake of blood pressure-lowering drugs

8.7: Rehabilitation sport with fluid deficiency

8.8: An additional intake of blood pressure-lowering drugs, but the blood pressure drops too much, probably due to an incorrect dose

8.9: Abnormalities during the night

FIG. 9

Representation of different device arrangements and their components for measuring the continuous course of the blood pressure using the improved Redtel method.

Variant 9.a consists only of a conventional blood pressure cuff (9.1) enhanced with the new logics of the improved Redtel method (9.2). The mode of operation is represented using the pressure course in FIG. 12.

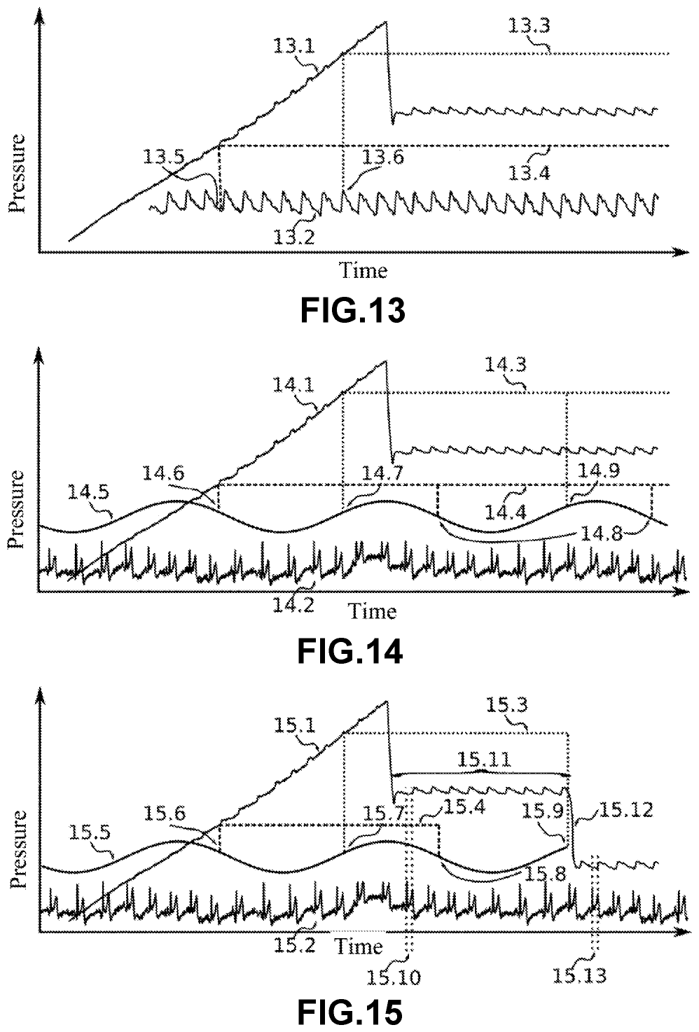

In variant 9.b, in addition, one or more pressure sensors (9.3) are introduced. These sensors are attached in the cuff so that they face the skin surface and are located over an artery; in a forearm cuff this artery is the radial artery, and in an upper arm cuff it is the brachial artery. The mode of operation is represented using the pressure course in FIG. 13.

In variant 9.c, the conventional blood pressure cuff is enhanced with an ECG (9.4). This ECG is used to improve the calibration of the improved Redtel method by the measured values based on the Riva-Rocci method. The exact design of the ECG can depend on the field of application. In a mobile variant, two electrodes can be inserted in the cuff facing the skin. These electrodes can consist, for example, of a stainless steel mesh. A third electrode can be attached on the housing of the control and display electronics. When the user touches this third electrode with a finger of the hand on the limb on which no measurement is carried out, the Cabrera circle is then closed and an ECG can be represented.

The use of wire-connected electrodes is also possible. In a mobile application, these electrodes are connected to the control electronics and they can be stuck on the body at appropriate sites. In the clinical application, the blood pressure cuff is attached to a patient monitor or connected to it; here the ECG signal is provided by the patient monitor, since these monitors as a rule have an ECG function. The mode of operation for the continuous representation of the pressure wave is represented using the pressure course in FIG. 14. An additional manner of use via the calibration of the blood pressure by the pulse transit time is represented in FIG. 15.

Variant 9.d is an enhanced form of variant [9.c, wherein this variant after calibration dispenses with an application pressure by the cuff and is therefore suitable for long-term measurement including over 24 hours. By use of a photo-plethysmography unit (9.5), the pulse course can be represented. Thus, the pulse transit time can be determined from the ECG signal (starting time) and from the plethysmography (end time). The pulse transit time by calibration can be used in order to determine the values of the blood pressure.

The plethysmography unit can be designed differently. A finger unit can be used; however, a unit in the blood pressure cuff, which is directed toward the skin surface, is also possible. Furthermore, a unit that is independent of the cuff is conceivable as a separate band around the arm.

The mode of operation is represented using the pressure course in FIG. 16.

Variant [9.e dispenses with an ECG and uses two photo-plethysmography units (9.5 and 9.6) instead. The plethysmography units can be designed as described in variant 9.d; however, the units should be attached separated from one another on the body. The spacing of the units with respect to one another results in a temporal offset in the measured value curves of the two units. In addition to this offset, the two measured value waves are deformed with respect to one another. This deformation is caused by the different velocities of the pulse wave for the diastole and the systole. The evaluation of the two measured value waves makes it possible to determine a pulse wave velocity for the diastole and a pulse wave velocity for the systole independently of one another. Thus, the diastole can be calibrated to the pulse transit time independently of the systole. The mode of operation is represented using the pressure course in FIG. 17.

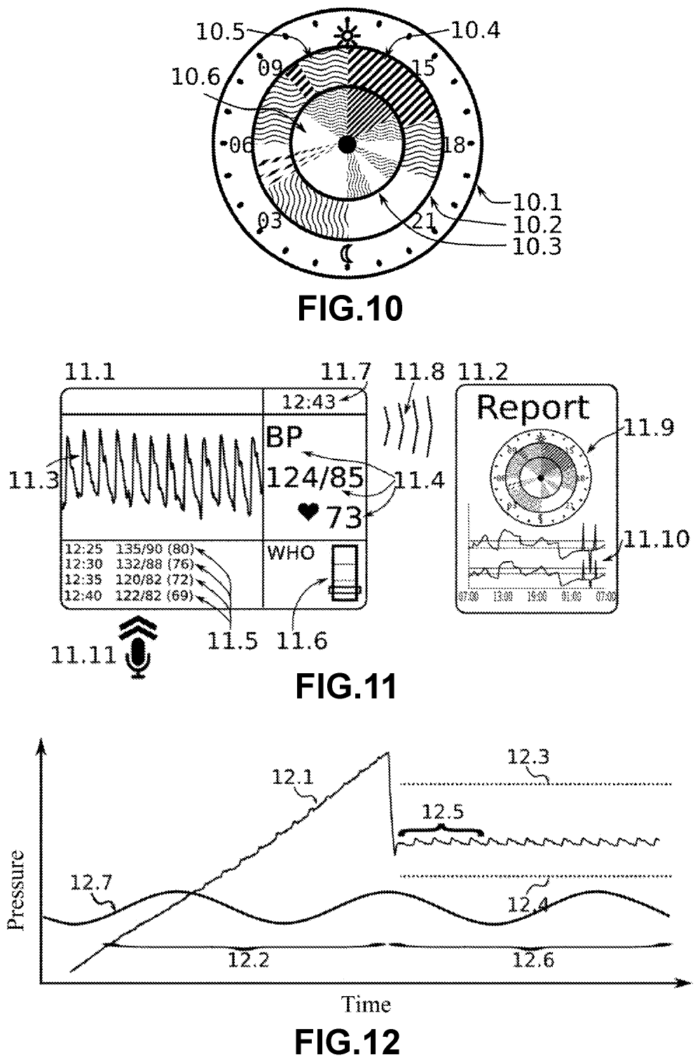

FIG. 10

An additional representation possibility for a diurnal profile (concerning FIG. 8). The goal of this representation is to represent critical and noncritical states in a single view. This representation possibility is particularly suitable as display in a clock or on the smartphone. This type of representation is suitable not only for the representation of the blood pressure but also for representing other types of diurnal profiles; however, here only the special application of blood pressure is discussed.

Shown is a concentric display which comprises different segments from outside to inside. In the case of the display of a blood pressure diurnal profile, these segments are preferably: a clock face of a 12- or 24-hour analog clock (10.1) for displaying the time of day, the course of the systole (10.2) and the course of the diastole (10.3).

The courses of systole and diastole are given by a coloring. The coloring depends on how critical the state is. For an evaluation according to WHO, this means that a healthy state exists if the value of the systole is lower than 120 mm Hg and the value of the diastole is lower than 80 mm Hg. An elevated blood pressure exists with blood pressure values between 120 and 140 for the systole and a diastole between 80 and 90 mm Hg. A pathological state is characterized by blood pressure values higher than 140 mm Hg for the systole or 90 mm Hg for the diastole. In the representation, healthy diurnal ranges are represented in green (here the non-crosshatched areas, for example, 10.6), elevated blood pressure ranges in the diurnal profile are represented in orange (here the wavy crosshatching, for example, 10.5), and ranges ranked as pathological are represented in red (here striped crosshatching, for example, 10.4). In a 12-hour clock, colors and/or concentric rings with different inner and/or outer diameters can designate a.m. and p.m.

FIG. 11

Representation possibility for the blood pressure and its course which was acquired according to the improved Redtel method, and user interaction possibility intended for the (interested) private user.

Shown is a display 11.1 which is either connected to a smartphone which works with a blood pressure cuff operated using the improved Redtel method per radio connection or which can be displayed directly on the display of a corresponding automatic blood pressure cuff machine.

Display 11.1 can represent the following components, inter alia:

11.3: Representation of the temporal course of the pressure in the arteries under the cuff.

11.4: Blood pressure value in the form of systole and diastole with a pulse rate for the current heartbeat.

11.5: Past blood pressure and pulse rate values with measurement times

11.6: Classification of the blood pressure, for example, according to the WHO criteria, on a color scale. Here either the current blood pressure of the current heartbeat or the totality of the measured values can be used for the classification.

11.7: Current time of day

In addition, it is possible to switch between display possibilities; thus, for example, the current diurnal profile can be displayed as represented, for example, in FIG. 10 or in FIG. 8.

The blood pressure measurement can moreover be controlled by a user interaction possibility (11.11). In the simplest case, this involves pressure buttons or touch fields on the display, but other control possibilities, for example, voice control, are also possible. In addition to the actual measurement control, this control also enables the entry of user comments. These comments are used for classifying a change in the diurnal profile. If the user is taking blood pressure-lowering drugs, this can be recorded in this way and marked accordingly in the diurnal profile. Other comments can relate, for example, to physical activity, therapy and medical measures, acute diseases, food intake, stress, discomfort, vertigo, pains or also sudden mood changes (for example, “got frightened”).

For further analysis by a medically trained expert, a report (11.2) can be generated automatically or upon input of the user. The report is deposited in a database or electronically transmitted (11.8), so that the expert has access to it.

This report can contain the following components, inter alia:

11.9: A diurnal profile in the form of a concentric representation, for example, as a clock (see also FIG. 10), so that the critical time segments can be seen in one view.

11.10: A detailed diurnal profile which represents the values for diastole and systole separately from one another for each time of the day and classifies them with color mark (see also FIG. 8). In addition, pressure course curves can be attached to the report, which show the beat-to-beat course (comparable to 11.3) and indicate an abnormality.

In addition to the simple display, output, and recording of the current state of the user, the user interaction possibility can also be used in order to output instruction signals to the user. These signals comprise, for example, the request to take medication (previously agreed on with a physician), to hydrate, to limit physical activity, to regulate food intake or other predefined actions. These instruction signals are output to the user based on the blood pressure situation and they can occur via sound, vibration, or visual representation on the display. Furthermore, information as a push message on the smartphone is possible.

FIG. 12

Typical air pressure course in a continuous blood pressure measurement with a conventional blood pressure cuff enhanced by the logics of the Redtel method but otherwise unmodified. Shown is the pressure course in the cuff as a function of time (12.1).

If a measurement is carried out, then first a measurement according to Riva-Rocci is carried out (time period 12.2). The measurement according to Riva-Rocci yields the values for the systole (12.3) and the diastole (12.4). With these values, in a calibration phase (12.5), scaling factors for obtaining a blood pressure curve from the air pressure in the cuff are determined. Subsequently, the temporal course of the air pressure values is used in order to be represented in scaled form as blood pressure curve. The calibration phase together with the representation phase (12.4) uses the Redtel method.

In an additional development stage, the respiration (12.7) can be determined from the pulse intervals. Here, the individual interval lengths are determined, which vary from pulse to pulse with the respiration, whereby the respiration is measurable. This can also occur during the Riva-Rocci phase, whereby the classification of the blood pressure values relative to the phase of the respiration is enabled (cf. description of related FIG. 14).

FIG. 13

Typical air pressure course and pressure course in a continuous blood pressure measurement with a conventional blood pressure cuff enhanced by the logics of the improved Redtel method, which also outputs, in addition to the measurement results, data on the time acquisition of these data. Shown is the air pressure course in the cuff (13.1) and the raw data of a pressure sensor (13.2). The measurement according to Riva-Rocci yields the values for the systole (13.3) and the values for the diastole (13.4). In addition to these values, the time of the collection is also output. These values can be used so that the values of the pressure sensor can be unequivocally calibrated. The calibration occurs at these times in the two curves (13.5 for the diastolic value and 13.6 for the systolic value).

The calibration is thus terminated at the end of the Riva-Rocci method.

FIG. 14:

One problem in the above-shown calibration methods is the assumption that the blood pressure is a constant value (at least briefly). This is not the case, particularly since the respiration leads to a variation of the blood pressure between each heartbeat, see FIG. 5.

In order to take into consideration the respiration, in addition to the air pressure curve (14.1), an ECG (14.2) is also recorded. The ECG can be examined with respect to the individual interval lengths and their difference with respect to one another. If the interval lengths are plotted as a function of time, then a wave can be detected (represented in idealized form as 14.5). The wavelength corresponds to a breath.

In the measurement according to Riva-Rocci, it is preferable to evaluate, in addition to the systolic value (14.3) and the diastolic value (14.4), their measurement times as well. These times are associated with a phase in the respiration (14.6 for the diastole and 14.7 for the systole). In the subsequent measurement according to the improved Redtel method, a calibration can then be carried out, when the respiration is in the same phase as in the measurements of diastole (for example, 14.8) and systole (for example, 14.9). This calibration makes it possible to compensate for the inaccuracy of the measurement according to Riva-Rocci due to the respiration, as described in FIG. 5.

Thus, this method can be used in order to carry out an individual or static measurement that is improved with respect to the conventional Riva-Rocci method. The measurement course would be exactly the same but the pressure is completely released after the calibration, and the measurement would be terminated. Since the calibration was carried out over a respiratory cycle, all the blood pressure values for the respiratory cycle are also known, and an indication of maximum and minimum values for diastole and systole is possible.

FIG. 15

In an additional development stage, the application pressure of the air pressure cuff is to be further reduced. Proceeding from the method described in FIG. 14 (points 15.1 to 15.9 correspond to points 14.1 to 14.9), the blood pressure is determined via the pulse transit time (for example, 15.10 or, for example, 15.13). A calibration of the pulse transit time relative to values of the blood pressure is only possible if different blood pressures are examined. This is given by the variation of the blood pressure due to the respiration. Therefore, in a calibration phase (15.11) which extends, for example, over a respiratory cycle, many or all of the values for the blood pressure are acquired and associated with the corresponding values for the respective heartbeat of the pulse transit time (for example, first heartbeat in the calibration phase 15.10). After the calibration phase, the pressure can be reduced (15.12). Subsequently, the pulse transit time is determined for each heartbeat (first heartbeat 15.13) and thus the blood pressure is calculated.

FIG. 16

The method in FIG. 15 cannot completely dispense with an application pressure. This is because the determination of the pulse transit time requires two curves of the activities of the heart, which are to be compared. The analysis of the ECG (16.2) yields the starting times. The end times were determined in FIG. 15 from the air pressure curve (15.1). In the case of a complete reduction of the air pressure, another curve of the activities of the heart would must also be recorded.

The method presented here is based on FIG. 15 (points 16.1 to 16.9 correspond to points 15.1 to 15.9).

For the determination of the end points for determining the pulse transit time, the data of a plethysmography unit (16.14) are used. The pulse transit times (for example, first heartbeat in the calibration phase 16.10 or, during the measurement, first heartbeat during the measurement 16.13) are then determined via the values of the ECG and of the plethysmography unit and thus correspond to the usual method. Here, the calibration is again possible due to the variation of the blood pressure due to the respiration. This method makes it possible to determine the blood pressure for each heartbeat, which also makes it possible to dispense with application pressure by the cuff.

FIG. 17

A greater accuracy in the determination of diastole and systole than in the method in FIG. 16 can be achieved if, instead of an ECG, the data of an additional plethysmography unit (17.2) are used. The method presented here is an enhancement of the method from FIG. 16, points 17.1, 17.3 to 17.9, 17.11, 17.12, 17.14 correspond to the respective points in FIG. 16.

The pulse transit time is then determined between the two data sets of the plethysmography units. Here, the transit time, for example, between the local minima or the local maxima can be determined.

Pulse transit times for the diastole and the systole result independently of one another, so that an independent calibration can also be carried out. The subsequently determined values for diastole and systole from the pulse transit time can thus also vary with respect to one another and independently of one another, in contrast to the current method (cf. FIG. 16).

FIG. 18

Representation of the spaced measurement by means of a camera. The spaced measurement makes it possible to simultaneously carry out at each point (for example, 18.1-18.3) of the body a measurement of the pulse wave velocity or pulse wave contour (18.4-18.6). In this example, there is an occlusive disease which can be detected in the measured value wave 18.5 since no pulse can be detected here.

FIG. 19

Representation of the spaced measurement. The spaced measurement can be used to carry out a comparative measurement. In this case, the right half of the face (19.1) and the left half of the face (19.2) are compared to one another. The pulse waves (19.3 and 19.4) in healthy humans have a temporal offset with respect to one another, which is in the range of 10 ms. In case of an occlusive disease or an early stage thereof, for example, a stenosis of the carotids, different temporal offsets can appear. The measurement of the offset can give indications of these diseases.

FIG. 20

Representation of the spaced measurement. An additional possibility for the detection of occlusive diseases is measurement on different extremities and comparison between the left and the right body halves. If a measurement is carried out between hand (20.1, 20.2) and foot (20.3, 20.4), the result is a temporal offset of the curves with respect to one another (20.5 with respect to 20.6, and 20.7 with respect to 20.8), due to the different lengths of arteries. However, in the healthy state, the result should be no difference between the left and right body halves. The offset between the measurements on the hands or respectively on the feet also should exhibit no offset or only a slight offset in the healthy state.

In both cases, a difference is an indication of a disease.

Aspects

The inventions can be described, for example, by means of the following aspects which can be combined individually or jointly with the following claims and/or aspects from the preceding description. Here, the device is configured for, in particular carrying out a method aspect.

1. Noninvasive continuous blood pressure measurement consisting of a combination of a pressurized and a non-pressurized continuous blood pressure measurement with a conventional blood pressure cuff device and an arrangement for measuring the pulse transit time or the pulse wave velocity, characterized in that the arrangement of these measurement systems enables an improved calibration to the blood pressure, which is free of human influence, in that the data of the individual methods are bundled in a computation unit.

2. Noninvasive continuous blood pressure measurement according to aspect 1, characterized in that, in the continuous blood pressure measurement, an air pressure buildup in a cuff is used for the detection of calibration values, wherein this measurement part is comparable to the Riva-Rocci method, and subsequently the air pressure is reduced and maintained, wherein these air pressure values are used for the continuous measurement of the blood pressure.

3. Noninvasive blood pressure measurement according to any one of the preceding aspects, characterized in that the measurement of the pulse transit time occurs with the help of devices for the determination of a starting time and an end time.

4. Noninvasive continuous blood pressure measurement according to any one of the preceding aspects, characterized in that the starting point and the end point are determined in each case with one or different ones of the following methods: with an ECG, with a continuous blood pressure measurement according to the “Redtel method,” with a plethysmography sensor, with a sensor for the oximetry measurement, with a pressure sensor, with an acoustic sensor, with a light intensity sensor, with a temperature sensor, with an impedance measurement device, or with a moving image camera.

5. Noninvasive continuous blood pressure measurement according to any one of the preceding aspects, characterized in that the pulse transit time or the pulse wave velocity is determined with a spaced measurement method which is based on the analysis of moving images or running video pictures.

6. Noninvasive continuous blood pressure measurement according to any one of the preceding aspects, characterized in that the pulse transit time or the pulse wave velocity is also determined by more than one plethysmography unit, wherein said units can also be located very close to one another, for example, with a spacing of 1 cm, when they use different light wavelengths; in the case of units that are at a distance from one another, for example, in the arm band and on the finger, the same light wavelength can be used.

7. Noninvasive continuous blood pressure measurement according to any one of the preceding aspects, characterized in that the pulse transit time is determined from the pulse wave contour.

8. Noninvasive continuous blood pressure measurement according to any one of the preceding aspects, characterized in that a position sensor is integrated.

9. Noninvasive continuous blood pressure measurement according to any one of the preceding aspects, characterized in that an acceleration sensor is integrated.

10. Noninvasive continuous blood pressure measurement according to any one of the preceding aspects, characterized in that the air pressure cuff used comprises a sensor for the determination of the arm diameter.

11. Noninvasive continuous blood pressure measurement according to aspect 10, characterized in that the sensor for the determination of the arm diameter is a bend sensor, an inductive sensor or a sensor which is based on capacitive touch technology.

12. Noninvasive continuous blood pressure measurement according to aspect 11, characterized in that the blood pressure cuff, in the case of the use of an inductive sensor or sensor based on “capacitive touch” technology, is closed stepwise.

13. Noninvasive continuous blood pressure measurement according to any one of aspects 10 to 12, characterized in that, at regular intervals, elements are introduced into the blood pressure cuff, which can be unequivocally identified by the sensor.

14. Noninvasive continuous blood pressure measurement according to any one of aspects 10 to 13, characterized in that an air sac of the air pressure cuff is subdivided into multiple chambers.

15. Noninvasive continuous blood pressure measurement according to aspect 14, characterized in that an active surface of the air sac of the air pressure cuff can be adapted to the arm diameter by connecting or disconnecting chambers by means of electrically switchable valves, wherein disconnected chambers are not filled with air during the measurement.

16. Noninvasive continuous blood pressure measurement according to aspect 14, characterized in that an active surface of the air sac of the air pressure cuff can be adjusted by only two chambers which are held on one another by belts, in that one chamber is used for the blood pressure measurement and the second chamber is used for the deformation of the first chamber, in that, by pressure change, the constriction of the first chamber by the belts is changed.

17. Noninvasive continuous blood pressure measurement according to any one of the preceding aspects, characterized in that the active surface of the air sac of an optimized air pressure cuff is also virtually changed and/or the air pressure cuff is designed so that this is possible.

18. Noninvasive continuous blood pressure measurement according to any one of the preceding aspects, characterized in that the data of the individual measurement systems are transmitted to at least one central computation unit, in particular via a wired connection, sound or via a radio connection, so that a temporal association of the individual data sets with one another is enabled.

19. Noninvasive continuous blood pressure measurement according to any one of the preceding aspects, characterized in that the data of the individual measurement systems follow at least one image, sound or vibration output.

20. Noninvasive continuous blood pressure measurement according to any one of the preceding aspects, characterized in that an arrangement is part of a medical monitor, this is particularly advantageous since such devices already have a plurality of suitable sensors and an easy integration is therefore possible.

21. Noninvasive continuous blood pressure measurement according to any one of the preceding aspects, characterized in that an output and control unit and/or computation unit is formed by a smart device which receives data per radio, sound or wire connection from an arrangement which uses the Redtel method, wherein the use of a smart device is advantageous since in many such devices a plurality of suitable sensors are already present: acceleration and position sensor, camera and light unit (flashlight), whereby a measurement similar to plethysmography is enabled, or even the moving image analysis with respect to the area-comprehensive pulse wave velocity is enabled.

22. Method for calibration of results of the measurement of the pulse transit time or pulse wave velocity in a living organism for obtaining continuous values of the blood pressure, characterized in that, for different blood pressure values of the living organism, the temporally associated pulse transit times or pulse wave velocities are known or have been collected.

23. Method for calibration according to aspect 22, characterized in that, for the calibration of the pulse transit time, the values of the blood pressure are acquired based on the continuous measurement using the “Redtel method.”

24. Method for calibration according to either aspect 22 or aspect 23, characterized in that, for each heartbeat, for, in particular each systole and/or diastole, values for the blood pressure and the pulse transit time or pulse wave velocity are determined.

25. Method for calibration according to any one of aspects 22 to 24, characterized in that the values of the measurement of the pulse transit time or pulse wave velocity are acquired at the same time as the values of the blood pressure.

26. Method for calibration according to any one of aspects 22 to 25, characterized in that the temporally associated values of the pulse transit time or pulse wave velocity and of the systolic or diastolic value of the blood pressure have a linear relationship, in particular of the form S_BD=A_s*PWG+B_s or D_BD=A_d*PWG+B_d, wherein S_BD is the systolic value, D_BD is the diastolic value, PWG is the pulse wave velocity, and A_s, B_s, A_d, B_d are the factors to be determined by the calibration.

27. Method for calibration according to any one of aspects 22 to 26, characterized in that the pulse wave velocity results from the pulse transit time, in that the interval between the measurement devices for starting time and end time is divided by the pulse transit time, but for the calibration it is sufficient to use the inverse of the pulse transit time instead of the pulse wave velocity.

28. Method for calibration according to aspect 27, characterized in that the factors A_s, B_s or A_d, B_d are determined from at least two temporally associated pairs of values of systolic blood pressure and pulse transit time or pulse wave velocity or from at least two temporally associated pairs of values of diastolic blood pressure and pulse transit time.

29. Method for calibration according to any one of aspects 22 to 28, characterized in that, for the calibration, the values of the blood pressure are different, wherein the value change is generated or can be generated due to a change in the elevation of the measurement site with respect to the HIP, but it is advantageous to use the natural variation due to the RSA for the calibration.

30. Method for calibration according to any one of aspects 22 to 29, characterized in that the frequency and the phase of the RSA and thus of the respiration measured can be determined or is determined based on the interval length by means of a system for the representation of heart functions, such as, for example, ECG, plethysmography, pressure sensors or the Redtel method.

31. Method for calibration according to any one of aspects 22 to 20, characterized in that the values of a conventional Riva-Rocci measurement can be determined for the phase of the respiration and other systems for the heart function representation, such as, for example, the Redtel method, are also calibrated in the phase of the respiration.

32. Method for calibration according to any one of aspects 22 to 31, characterized in that, taking into consideration the respiration, the calibration is also used for the improvement of the conventional Riva-Rocci measurement in the individual value determination and thus a value is determined, which includes the respiration, a possible indication of a value is, for example, highest systole, lowest diastole, greatest pulse pressure in the respiration or systole and diastole with respect to a fixed aspect within the phase of the respiration, for example, in the fully inhaled state.

33. Method for calibration according to any one of aspects 22 to 32, characterized in that, when methods reproducing pulse pressure are used, such as, for example, air pressure cuffs, plethysmography units or pressure sensors, the pulse transit time, for different parts of the pulse pressure wave such as, for example, the diastole, the systole or the reflection wave, are determined independently of one another and in particular are calibrated independently of one another to the blood pressure.

34. Method for calibration according to any one of aspects 22 to 33, characterized in that different methods for the representation of the pulse pressure wave for the analysis of the pulse transit time are tuned to one another, wherein this occurs by the use at the same site on the body, by tuning with a verified method or, in the case of light-based methods, by tuning with a color pattern map.

35. Method for calibration of results of the measurement according to any one of aspects 22 to 34, wherein a measurement of the variable light intensity for the obtention of values of the variable blood pressure occurs so that a continuous measurement of the blood pressure is enabled, characterized in that additionally and in particular a simultaneous measurement is carried out first with a continuous measurement device working using the “Redtel method” and with a plethysmography sensor, and subsequently, without the measurement device working according to the “Redtel method” but only with the values of a plethysmography sensor or of a light intensity sensor after calibration, the blood pressure curve is represented.

36. Method for calibration according to any one of aspects 22 to 35, characterized in that the waves of plethysmography sensor and of the measurement device working using the “Redtel method” behave linearly with respect to one another but are shifted temporally by the pulse transit time between the measurement devices, whereby the values of the plethysmography sensor or light intensity sensor can be calibrated, in particular linearly, by: P(t-PWL)=C*L(t)+D, wherein P(t-PWL) is the pressure in the artery at the time when this pressure point passed the site of the cuff and L(t) is the light intensity at the measurement time, C and D are the parameters resulting during the calibration from a linear approximation.

37. Method for calibration according to any one of aspects 22 to 36, characterized in that the voltages present in the ECG signal due to functions of the heart can be used for the determination of the blood pressure.

38. Method for calibration according to any one of aspects 22 to 37, characterized in that the voltages present in the ECG signal due to the functions of the heart is determined for each pulse or heartbeat in one or more respiratory cycles.

39. Method for calibration according to any one of aspects 22 to 38, characterized in that the voltages present in the ECG signal due to functions of the heart is determined at the same points in the respiratory cycle.

40. Method for calibration according to any one of aspects 22 to 39, characterized in that the voltages which are present in the ECG signal due to functions of the heart change with the blood pressure from one breath to the next, this change being used in order to calibrate them to the blood pressure using the values of the blood pressure at the same time in the breath.

41. Method for calibration according to any one of aspects 22 to 40, characterized in that the voltages present in the ECG signal due to functions of the heart, after calibration, enable the determination of blood pressure values also without Redtel method.

42. Method for application of a noninvasive continuous blood pressure measurement according to any one of aspects 1 to 21, consisting of a combination of a pressurized continuous blood pressure measurement with a conventional blood pressure cuff device and an arrangement for the measurement of the pulse transit time or of the pulse wave velocity, characterized in that the pressurized blood pressure measurement, the blood pressure curves are measured peripherally, for example, on the extremities, such as thigh or calf, toes, feet but in particular and advantageously also on the upper arm, wrist or finger and/or also with a camera spaced on freely accessible skin surfaces.

43. Method of application according to aspect 42, characterized in that, after calibration, the pressurization is relaxed, whereby, in particular, no impairment of the lymph or of the venous systems occurs and an unlimited continuous blood pressure measurement can occur.

44. Method of application according to either aspect 42 or aspect 43, characterized in that the invention is suitable for the preventive, rehabilitative, ambulatory and clinical use.

45. Method of application according to any one of aspects 42 to 44, characterized in that the pulse transit time or pulse wave velocity is determined with a simple arrangement consisting of a smartphone and a blood pressure cuff which works according to the “Redtel method.”

46. Method of application according to any one of aspects 42 to 45, characterized in that, by comparative measurements on the extremities, information can be obtained regarding possible existing or incipient arterial occlusions—stenosis and/or arteriosclerosis, wherein there is an indication that the average pulse transit time or pulse wave velocity differs from the right to the left extremity.

47. Method of application according to any one of aspects 42 to 46, characterized in that, by the analysis of pictures or moving images, the pulse transit time is acquired area-comprehensibly and continuously for each heartbeat over the body section acquired in the pictures, so that sites which are weaker than usual or not pulsating are made visible, wherein these sites are possibly affected by or are beginning to develop vessel stiffness due to plaque deposits or even occlusive diseases such as, for example, arterial occlusions-stenoses, as well as arteriosclerosis or also aesthetic abnormalities such as, for example, varicose veins or orange skin.

48. Method of application according to any one of aspects 42 to 47, characterized in that, by the analysis of pictures or moving images, the pulse transit time can also be determined for, in particular individual toes or fingers.

49. Method of application according to any one of aspects 42 to 48, characterized in that differences in the pulse transit time of one or more toes or fingers with respect to one another can be detected on one member and thus constitute a sign of a possible pathologically worsened blood circulation, which is a frequent clinical picture, for example, in diabetes.

50. Method of application according to any one of aspects 42 to 49, characterized in that the measurement artifacts due to a changed position with respect to the HIP in connection with the calibration are detected by a position sensor or an acceleration sensor.

51. Method of application according to any one of aspects 42 to 50, characterized in that a changed position with respect to the HIP in connection with the calibration is determined by a position sensor or an acceleration sensor, in particular is determined by previous calibration, so that the value of the blood pressure is determined with knowledge of the position with respect to the HIP.

52. Method of application according to any one of aspects 42 to 51, characterized in that, from the course of the continuous blood pressure, the vital state and the mental state are derived, in particular in order to prompt or control autonomous actions for safeguarding the person, for example, in intensive care, or for the control of devices and installations such as, for example, the output of haptic and automated medication and nutrition.

53. Method of application according to any one of aspects 42 to 52, characterized in that the respiration is detected based on changes of the pulse rate during the RSA in order to provide objective justification in the field of alternative medicine as well.

54. Method of application according to any one of aspects 42 to 53, characterized in that a simple medical validation is possible, since, for the individual components, medically validated versions are already available and thus nothing stands in the way of their introduction for use in the daily clinical routine, whereby the stress on the patient can be reduced due to the omission of the invasive blood pressure measurement.

55. Method of application according to any one of aspects 42 to 54, characterized in that the elevation of the measurement device with respect to the HIP is acquired and used in particular to indicate the blood pressure at the elevation of the HIP, wherein the pressure difference (dP_d diastolic and dP_s systolic pressure difference) of the measurement site due to the elevation difference dH with respect to the HIP is obtained as follows:

dP_d=a_d*dh and dP_s=a_s*dH, wherein the factors a_d and a_s are obtained empirically and at the time are assumed to be a_d=0.5 mm Hg/cm and a_s=1 mm Hg/cm.

56. Method of application according to any one of aspects 42 to 55, characterized in that the elevation of the measurement device with respect to the HIP is determined in particular by a calibration by means of predetermined movement of the measurement device by the user.

57. Method of application according to any one of aspects 42 to 56, characterized in that the elevation of the measurement device with respect to the HIP can also be determined in that an additional device which transmits its orientation to the control unit is used and it enables the determination of the distance from the measurement device.

58. Method of application according to any one of aspects 42 to 57, characterized in that the elevation of the measurement device with respect to the HIP can also be determined in that only the orientation of the HIP and of the measurement device with respect to the surface of the earth is determined and is evaluated in combination with the measurement position on the body, for example, the arm, and its length.

59. Method of application according to any one of aspects 42 to 58, characterized in that the collected vital data and/or blood pressure values, in particular of a long-term measurement, are represented in a diurnal profile.

60. Method of application according to any one of aspects 42 to 59, characterized in that the physical state, state of health, or psychological state and also states defined by the user are detected based on the variations of blood pressure, RR interval and respiration and are indicated in particular by warning messages, and this can occur in particular in connection with the generation of a diurnal profile which represents the course of the determined vital data.

61. Method of application according to any one of aspects 42 to 60, characterized in that the continuous monitoring of the state of health, of the psychological state and of the physical state can be used in order to control the daily routine of a user, by instructions during physical activity, by nutrition and drinking suggestions, by suggestions, for example, in the case of cardiovascular diseases, respiratory diseases, sleep-related breathing disorders, diabetes, by suggestions for improving the stress level, or preferably indications concerning medication.

62. Method of application according to any one of aspects 42 to 61, characterized in that, in the case of continuous recording of vital data, for example, in the context of a diurnal profile, these data are checked for changes while they are recorded, whereby, by means of the measurement device, in case of critical changes or user-defined changes, warnings are output to the user by acoustic means, on a display, by vibrations or by transmitting push messages on a smartphone, or are transmitted electronically to another device.

63. Method of application according to any one of aspects 42 to 62, characterized in that, by means of the warnings, before critical changes, the drug intake of the user can be regulated, so that the drug intake tailored to the change takes place only when the medication is necessary.

64. Method of application according to any one of aspects 42 to 63, characterized in that, due to the possibility of tailored medication, suitable products of the pharmaceutical industry can be created, which enable smaller dose quantities but multiple medication intakes automatically or manually distributed over the day.

65. Method of application according to any one of aspects 42 to 64, characterized in that, due to the warnings before critical changes, devices can be controlled or they can be adapted to the situation; these devices can be, for example, automated medication systems, respirators, emergency call systems or transport means which can then transmit an automated emergency call.

1. A method for, blood pressure measurement, comprising a combination of:
at least two pressurized blood pressure measurements for different respiratory states or elevations of the measuring point with respect to the heart, and at least two non-pressurized measurements of the pulse wave velocity, of the pulse wave contour, of the electrical activity of the heart or of the blood pressure;
wherein the pressurized and the non-pressurized measurements are carried out on a same living organism;
wherein later non-pressurized measurements of the blood pressure, of the pulse transit time, of the pulse wave velocity or of the pulse wave contour are carried out, and measured values of the later non-pressurized measurements are converted by means of data collected in the pressurized blood pressure measurement into at least one blood pressure value.

2. A device or system for blood pressure measurement, comprising:
means for carrying out a non-pressurized continuous measurement of a pulse transit time, of a pulse wave velocity, of a pulse wave contour, of electrical activity of a heart or blood pressure of an organism, wherein the device is configured;
to receive measured values of at least two pressurized blood pressure measurements for different respiratory states or elevations of a measurement point with respect to the heart, to jointly process the received measured values of the at least two pressurized blood pressure measurements and non-pressurized measurements for calibration purposes; and
to carry out numerous additional non-pressurized measurements of the blood pressure, of the pulse transit time, of the pulse wave velocity or of the pulse wave contour, and to convert the numerous additional non-pressurized measurements of the blood pressure, of the pulse transit time, of the pulse wave velocity or of the pulse wave contour in each case by means of the calibration obtained into in each case at least one blood pressure value and to output said at least one blood pressure value.

3. The device or system according to claim 2, further comprising a means for acquisition and output of the at least two pressurized blood pressure measurements for different respiratory states or different elevations of the measurement point of the blood pressure measurement with respect to the heart.

4. A method for calibrating results of measurement of a pulse transit time, a pulse wave velocity, a pulse wave contour or of electrical activity of a heart in a living organism for obtaining continuous values of blood pressure, wherein for at least one blood pressure value a percentage of inspiration or expiration or a temporal position in the respiratory cycle, the pulse transit time, the pulse wave velocity, the pulse wave contour or the electrical activity of the heart, which are associated with respective blood pressure values, are used or collected.

5. A use of an influence of respiration or of different elevations of a measurement point of a blood pressure measurement with respect to a heart on a blood pressure for a calibration of a measurement of a pulse transit time, of a pulse wave velocity, of a pulse wave contour or electrical activity of the heart, for computation of a blood pressure from measured values of the pulse transit time, the pulse wave velocity, the pulse wave contour or the electrical activity of the heart.

6. (canceled)

7. A method, for blood pressure measurement, wherein, by means of at least one pressure transducer, a pressure variation caused by blood pressure is continuously acquired, wherein at least two blood pressure values are determined from the acquired pressure variations, wherein an influence of respiration on determined blood pressure values is reduced, in that, from the continuously acquired blood pressure variations, the influence of the respiration on the variation is determined or respiratory states are determined and the at least blood pressure values are derived from the values acquired by the at least one pressure transducer, which were acquired for a predetermined or identical respiratory state.

8. A device, for blood pressure measurement, comprising:
at least one pressure transducer for continuous acquisition of a pressure variation caused by blood pressure;
wherein the device is configured to determine and output at least two blood pressure values from the continuously acquired pressures pressure variations;
wherein the device is configured to reduce an influence of respiration on the determined at least two blood pressure values, in that the device determines the influence of the respiration on a variation or respiratory state from the continuously acquired pressure variations and derives the at least two blood pressure values from the values acquired by the pressure transducer which were acquired for a predetermined or identical respiratory state.

9. The method according to claim 1, wherein the pulse transit time, the pulse wave velocity, the pulse wave contour or the electrical activity of a heart for different parts of a pulse pressure wave are determined independently of one another, are used or calibrated to the blood pressure, or wherein the pulse transit time, the pulse wave velocity, the pulse wave contour or the electrical activity of the heart is determined by means of an air pressure cuff, a plethysmography unit, an Electrocardiogram (ECG) or a pressure sensor.

10. The method according to claim 9, wherein the pressurized calibration measurements or blood pressure measurements and non-pressurized calibration measurements occur temporally close together, simultaneously or for a similar respiratory state or wherein the pressurized calibration measurements or blood pressure measurements and non-pressurized calibration measurements occur at different points on a body of a living organism, wherein the different points are selected so that a blood vessel extending from or to the heart successively reaches the different points.

11. The method according to claim 10, wherein, after a calibration, the pressurization is relaxed and further non-pressurized measurements, are carried out, for at least 30 minutes.

12. The method according to claim 10, wherein a change in a position of a measurement point with respect to a Hydrostatic Indifference Point (HIP) or to the heart is acquired by a position or acceleration sensor and used for correction of the measurements.

13. The method according to claim 1, wherein the pulse transit time is determined from the pulse wave contour.

14. The method device, according to claim 8, wherein, for the pressurized blood pressure measurement, an air pressure cuff is used, which comprises a sensor for a determination of an arm diameter, wherein the air pressure cuff is closed stepwise or elements are introduced into the air pressure cuff at regular intervals, which are unequivocally identified by the sensor, or in that an air sac of the air pressure cuff is subdivided into multiple chambers, and an active surface of the air sac of the air pressure cuff is adapted to the arm diameter by connecting or disconnecting chambers by means of electrically switchable valves, wherein non-connected chambers are not filled with air during the measurement or the air pressure cuff is designed so that the active surface of the air sac of the air pressure cuff is adjusted by two chambers which are held together, in that a first of the two chambers is used for the blood pressure measurement and a second of the two chambers is used for deformation of the first chamber.

15. The method according to claim 1, wherein, on a basis of measurements of cardiac activity, devices are controlled or control instructions or handling instructions are output, wherein the devices are automated medication systems configured to transmit an automated emergency call, or are autonomously driving transport means which autonomously react, in that, a warning is output to a user or, an emergency call is triggered, or the autonomously driving transport means is driven onto a roadside or a trip to a hospital is initiated, wherein, a permitted maximum speed is exceeded, which is low-risk by signaling of the autonomously driving transport means to other traffic participants, by light, sound or radio signals.

16. The method according to claim 1, wherein a detection of a respiratory state, of the respiration or of respiration frequency occurs by acquisition of periodic changes of diastole and systole, an interval or pattern of the diastole and the systole; or
by acquisition of periodic changes of a pulse pressure; or
by acquisition of periodic changes of a respiratory rate (RR) interval or
by acquisition of periodic changes of oxygen content in the blood.

17. A method for lifesaving or protection of traffic participants, wherein, on a basis of measurements of cardiac activity of a passenger of a transport means by measurement of blood pressure or a pulse, wherein means used for the measurement communicate with the transport means, and when critical heart states are detected, an emergency call is transmitted by the transport means or a warning is issued to a user or an emergency call is triggered or the transport means is driven to a shoulder of a traffic lane, or a trip to a hospital is initiated or carried out, wherein, a permitted maximum speed is exceeded, which is designed to be low risk by signaling the transport means to other traffic participants, by light, sound or radio signals.

18. A transport means configured to communicate with a means for measurements of cardiac activity of a passenger of the transport means, wherein the measurements of cardiac activity include measurements of blood pressure or a pulse, wherein the transport means is configured so that, when critical heart states are detected, the transport means transmits an emergency call or outputs a warning to a user or drives to a shoulder of a traffic lane or initiates or carries out a trip to a hospital wherein the transport means exceeds a permitted maximum speed, which is designed to be low-risk, by signaling the transport means to other traffic participants by light, sound or radio signals.
